(19) United States
(12) Patent Application Publication
(10) Pub. No.: US 2006/0287548 A1
(43) Pub. Date: Dec. 21, 2006

(54) Radical interceptors as stabilizers of polymerizable compounds

(76) Inventors: Frank Hoefer, Bad Duerkheim (DE); Sylke Haremza, Neckargemuend (DE); Gerhard Wagenblast, Wachenheim (DE); Volker Schliephake, Schifferstadt (DE); Ulrich Jaeger, Roemerberg (DE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 10/573,472

(22) PCT Filed: Sep. 18, 2004
(86) PCT No.: PCT/EP04/10492

§ 371 Date: Mar. 24, 2006

(30) Foreign Application Priority Data
Sep. 26, 2003 (DE) 03021803.6
Oct. 1, 2003 (DE) 10346135.3

(51) Int. Cl. C07C 69/02

(52) U.S. Cl. 560/4

(57) ABSTRACT

Process for stabilizing polymerizable compounds to polymerization during working-up, storage and/or transport, in which at least one free radical scavenger which contains at least two glycine units is used, with the proviso that the free radical scavenger is not selected from ethylenediaminetetraacetic acid (EDTA), diethylenetriaminepentaacetic acid (DTPA), trans-1,2-cyclohexanediaminetetraacetic acid (CYDTA) and the alkali metal and alkaline earth metal salts thereof.

The present invention describes the use of free radical scavengers for stabilizing polymerizable compounds to polymerization.

It is known that polymerizable compounds can readily be caused to polymerize, for example by heat or the action of light or peroxides. Since, however, polymerization has to be diminished or reduced for safety and economic reasons during production, working-up, storage and/or transport, there is a constant demand for novel, effective polymerization inhibitors.

Particularly problematic are the chemical and/or physical processing during the working-up, for example by distillation or rectification, and subsequently the storage and the transport.

A large number of stabilizers for polymerizable compounds are known, in particular for acrylic acid and methacrylic acid, referred to below as (meth)acrylic acid, and the esters thereof, referred to below as (meth)acrylates.

GB-A 1 601 979 describes the stabilization of an aqueous solution of a (meth)acrylate salt with a nitrosophenolate in the presence of EDTA as a chelator.

U.S. Pat. No. 4,929,660 discloses an adhesive composition which contains a free radical acrylic monomer and a polymerization inhibitor, a metal chelator and a free radical scavenger. This free radical scavenger is an N,N-dialkyl- or N,N-diarylalkylhydroxylamine.

The stabilization of unsaturated quaternary ammonium salts in the presence of metal scavengers is described in U.S. Pat. No. 5,912,383. These metal scavengers may be diethylenetriaminepentaacetic acid and N-(hydroxyethyl)ethylenediaminetriacetic acid and the associated sodium salts.

DE-A 199 20 796 describes a process for the preparation of isobornyl (meth)acrylates by reacting camphene with (meth)acrylic acid, which process is likewise carried out in the presence of a chelate former. Nitrilotriacetic acid, ethylenediaminetetraacetic acid, N-(2-hydroxyethyl)ethylenediaminetriacetic acid, 1,2-cyclohexylenedinitrilotetraacetic acid, diethylenetriaminepentaacetic acid, 3,6-dioxaoctamethylenedinitrilotetraacetic acid and the alkali metal salts of these acids are disclosed as chelate formers in this publication.

WO 02/26685 describes the stabilization of acrylic monomers during the distillation with a stabilizer in the presence of oxygen with a metal scavenger which is selected from ethylenediaminetetraacetic acid (EDTA), diethylenetriaminepentaacetic acid (DTPA) and the sodium salt thereof (Na₅DTPA) and trans-1,2-cyclohexanediaminepentaacetic acid. These metal scavengers are used for complexing free iron.

JP 05-295011 and JP 05-320205 likewise describe the stabilization of acrylic acid in the presence of EDTA, DTPA, CYDTA and the alkali metal salts thereof.

It is an object of the present invention to provide an alternative process for stabilizing polymerizable compounds to polymerization during working-up, storage and/or transport.

We have found that this object is achieved by a process for stabilizing polymerizable compounds to polymerization during working-up, storage and/or transport, where at least one free radical scavenger which contains at least two glycine units is used, with the proviso that the free radical scavenger is not selected from ethylenediaminetetraacetic acid (EDTA), diethylenetriaminepentaacetic acid (DTPA), trans-1,2-cyclohexanediaminetetraacetic acid (CYDTA) and the alkali metal and alkaline earth metal salts thereof.

In a preferred embodiment, at least one free radical scavenger which contains at least two glycine units is used, with the proviso that the free radical scavenger does not have ≧2 of the following structural units: 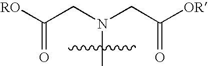
where R and R′, independently of one another, may be hydrogen or metal. For example, these metals may be alkali metals, such as sodium or potassium.

In a particularly preferred embodiment, at least one free radical scavenger which contains at least two glycine units and at least one amide and/or ester unit is used. The free radical scavenger preferably has two amide units.

Very particularly preferably used free radical scavengers are those of the formula (I):

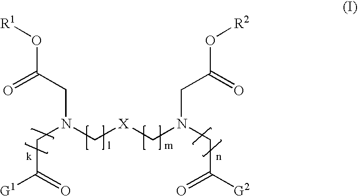

Specifically:

G¹ may be NR³R⁴ or OR⁷ and G² may be NR⁵R⁶ or OR⁸.

X may be C₁-C₂₀-alkyl, NCH₂COOR⁹, NR¹⁰, O, S, PR¹¹, Se, SiOR¹²R¹³ or aryl, where said substituents may be substituted in any desired position, but not more than five times, preferably not more than four times, particularly preferably not more than three times, by one or more hetero atoms and/or halogen atoms. X is preferably a C₁-C₂₀-alkyl group or NCHCOOR⁹, particularly preferably X is a C₁-C₁₀-alkyl group or NCHCOOR⁹.

k, l, m and n, independently of one another, are from 0 to 20. l and m are preferably in the range from 0 to 10, particularly preferably from 0 to 5, very particularly preferably from 0 to 3 and particularly preferably from 0 to 2. k and n preferably have the same value, e.g. in the range from 0 to 10, preferably from 0 to 5, very particularly preferably from 1 to 3; in particular k and n have the value 1.

Specifically, the radicals R¹ to R⁸ may have the following meaning:

R¹ to R⁶, independently of one another, may be hydrogen, C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₅-C₁₅-cycloalkylcarbonyl, aryl, arylcarbonyl or heterocycles,

R⁷ and R⁸, independently of one another, may be C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₅-C₁₅-cycloalkylcarbonyl, aryl, arylcarbonyl or heterocycles,

where

a) in the case of said aliphatic substituents, the radicals R³ and R⁴ or R⁵ and R⁶ may also be linked to one another and together may thus form a three-membered to eight-membered, preferably a five-membered to seven-membered and particularly preferably a five-membered or six-membered ring,
b) said aliphatic substituents may be straight-chain or branched,
c) the substituents may each be interrupted in any desired position by one or more hetero atoms, the number of these hetero atoms being not more than 10, preferably not more than 8, particularly preferably not more than 5 and in particular not more than 3, and/or
d) the substituents may each be substituted in any desired position, but not more than five times, preferably not more than four times and particularly preferably not more than three times, by alkyl, alkyloxy, alkyloxycarbonyl, aryl, aryloxy, aryloxycarbonyl, hydroxycarbonyl, aminocarbonyl, heterocycles, hetero atoms or halogen atoms, it being possible for these likewise to be substituted not more than twice, preferably not more than once, by said groups.

R⁹ to R¹³, if appropriate independently of one another, are hydrogen or C₁-C₂₀-alkyl.

Specifically, the general terms stated for the various radicals R have the following meaning:

C₁-C₂₀-Alkyl: straight-chain or branched hydrocarbon radicals of up to 20 carbon atoms, preferably C₁-C₁₀-alkyl, such as methyl, ethyl, propyl, isopropyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 2,4,4-trimethylpentyl, 1,1,3,3-tetramethylbutyl, nonyl and decyl and the isomers thereof.

C₁-C₂₀-Alkylcarbonyl: a straight-chain or branched alkyl group of 1 to 20 carbon atoms (as stated above) which are attached via a carbonyl group (—CO—), preferably C₁-C₁₀-alkylcarbonyl, for example formyl, acetyl, n-propionyl or isopropionyl, n-butanoyl, isobutanoyl, sec-butanoyl or tert-butanoyl, n-pentanoyl, isopentanoyl, sec-pentanoyl or tert-pentanoyl, n-nonanoyl or isononanoyl or n-decanoyl.

C₂-C₂₀-Alkenyl: unsaturated, straight-chain or branched hydrocarbon radicals having 2 to 20 carbon atoms and a double bond in any desired position, preferably C₂-C₁₀-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-Trimethyl-2-propenyl, 1-ethyl-1-methyl-2-propenyl, 1-ethyl-2-methyl-1-propenyl and 1-ethyl-2-methyl-2-propenyl and the isomers of heptenyl, octenyl, nonenyl and decenyl.

C₂-C₂₀-Alkenylcarbonyl: unsaturated, straight-chain or branched hydrocarbon radicals having 2 to 20 carbon atoms and a double bond in any desired position (as stated above) which are attached via a carbonyl group (—CO—), preferably C₂-C₁₀-alkylcarbonyl, for example ethenoyl, propenoyl, butenoyl, pentenoyl, nonenoyl and the isomers thereof.

C₂-C₂₀-Alkynyl: straight-chain or branched hydrocarbon radicals having 2 to 20 carbon atoms and a triple bond in any desired position, preferably C₂-C₁₀-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the isomers of heptynyl, octynyl, nonylyl and decynyl.

C₂-C₂₀-Alkynylcarbonyl: unsaturated, straight-chain or branched hydrocarbon radicals having 2 to 20 carbon atoms and a triple bond in any desired position (as stated above) which are attached via a carbonyl group (—CO—), preferably C₂-C₁₀-alkynylcarbonyl, for example propynoyl, butynoyl, pentynoyl, nonynoyl, decynoyl and the isomers thereof.

C₃-C₁₅-Cycloalkyl: monocyclic, saturated hydrocarbon groups having 3 to 15 carbon ring members, preferably C₃-C₈-cycloalkyl, such as cyclopropyl, cyclobutyl, cyclopentyl, cyclohexyl, cycloheptyl and cyclooctyl and a saturated or unsaturated cyclic system, such as norbornyl or norbenyl.

C₃-C₁₅-Cycloalkylcarbonyl: monocyclic, saturated hydrocarbon groups having 3 to 14 carbon ring members (as stated above) which are attached via a carbonyl group (—CO—), preferably C₃-C₈-cycloalkylcarbonyl.

Aryl: a mononuclear to trinuclear aromatic ring system comprising 6 to 14 carbon ring members, e.g. phenyl, naphthyl and anthracenyl, preferably a mononuclear or dinuclear, particularly preferably a mononuclear, aromatic ring system.

Arylcarbonyl: preferably a mononuclear to trinuclear aromatic ring system (as stated above) which is attached to the frame via a carbonyl group (—CO—), such as benzoyl, preferably a mononuclear or dinuclear, particularly preferably a mononuclear, aromatic ring system.

Heterocycles: ring system having, if appropriate, a plurality of five-membered to twelve-membered, preferably five-membered to nine-membered, particularly preferably five-membered or six-membered rings having oxygen, nitrogen and/or sulfur atoms, for example furyl, thiophenyl, pyrryl, pyridyl, indolyl, benzooxazolyl, dioxolyl, dioxyl, benzimidazolyl, benzothiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl.

The substituents mentioned specifically may, as described above, each be interrupted in any desired position by one or more hetero atoms, the number of these hetero atoms being not more than 10, preferably not more than 8, very particularly preferably not more than 5 and in particular not more than 3, and/or may each be substituted in any desired position, but not more than five times, preferably not more than four times and particularly preferably not more than three times, by alkyl, alkoxy, alkoxycarbonyl, aryl, aryloxy, aryloxycarbonyl, hydroxycarbonyl, aminocarbonyl, heterocycles, hetero atoms or halogen atoms, it being possible for these likewise to be substituted not more than twice, preferably not more than once, by said groups.

The classes of compounds, alkyl, aryl and heterocycles, mentioned in this group have the abovementioned meaning.

Hetero atoms are oxygen, nitrogen, sulfur or phosphorus.

Alkoxy is a straight-chain or branched alkyl group of 1 to 20 carbon atoms (as stated above) which is attached to the frame via an oxygen atom (—O—), preferably C₁-C₁₀-alkoxy, such as methoxy, ethoxy or propoxy.

Alkoxycarbonyl is an alkoxy group of 1 to 20 carbon atoms (as stated above) which is attached to the frame via a carbonyl group (—CO—), preferably C₁-C₁₀-alkoxycarbonyl.

Aryloxy is a mononuclear to trinuclear aromatic ring system (as stated above) which is attached to the frame via an oxygen atom (—O—), preferably a mononuclear or dinuclear, particularly preferably a mononuclear, aromatic ring system.

Aryloxycarbonyl is a mononuclear to trinuclear aryloxy group (as stated above) which is attached to the frame via a carbonyl group (—CO—), preferably a mononuclear or dinuclear, particularly preferably a mononuclear, aryloxycarbonyl.

Halogen atoms are fluorine, chlorine, bromine and iodine.

The radicals R¹ and R² are preferably identical and are hydrogen or C₁-C₂₀-alkyl, particularly preferably hydrogen or C₁-C₁₀-alkyl, very particularly preferably hydrogen or C₁-C₆-alkyl.

The radicals R³ and R⁵ are preferably identical and are hydrogen, C₁-C₂₀-alkyl or C₁-C₂₀-alkylcarbonyl, particularly preferably hydrogen, C₁-C₁₀-alkyl or C₁-C₁₀-alkylcarbonyl, very particularly preferably hydrogen, C₁-C₆-alkyl or C₁-C₆-alkylcarbonyl.

The radicals R⁴ and R⁶ or R⁷ and R⁸ are preferably identical and are C₁-C₂₀-alkyl, C₁-C₂₀-alkylcarbonyl, aryl, C₂-C₂₀-alkenyl, C₂-C₂₀-alkenylcarbonyl, C₂-C₂₀-alkynyl or C₂-C₂₀-alkynylcarbonyl. As described above, the radicals may each be interrupted in any desired position by one or more hetero atoms, the number of these hetero atoms being not more than 10, preferably not more than 8, very particularly preferably not more than 5 and in particular not more than 3, and/or may be substituted in any desired position, but not more than five times, preferably not more than four times and particularly preferably not more than three times, by alkyl, alkoxy, alkoxycarbonyl, aryl, aryloxy, aryloxycarbonyl, hydroxycarbonyl, aminocarbonyl, heterocycles, hetero atoms or halogen atoms, it being possible for these likewise to be substituted not more than twice, preferably not more than once, by said groups.

Particularly preferred radicals R⁴ and R⁶ or R⁷ and R⁸ are selected from phenyl, benzyl, p-methoxyphenyl, o-, m- or p-hydroxyphenyl, 1-hydroxyhexyl, methyl, ethyl, propyl, butyl, ethylene glycol, diethylene glycol, triethylene glycol, ethoxylate having from 4 to 10 EO units, ethylenediamine, diethylenetriamine, triethylenetetramine and amino acids, such as alanine, valine, leucine, isoleucine, proline, tryptophan, phenylalanine, methionine, glycine, serine, tyrosine, threonine, cysteine, asparagine, glutamine, aspartic or glutamic acid, lysine, arginine or histidine.

For example, R⁴ and R⁵ or R⁷ and R⁸ may be the radicals mentioned in table 1.

TABLE 1

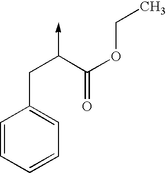

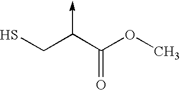

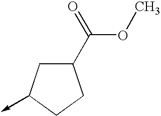

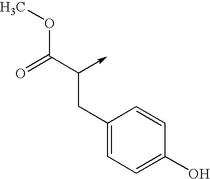

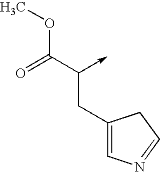

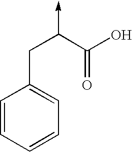

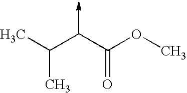

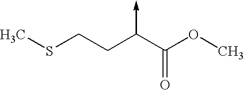

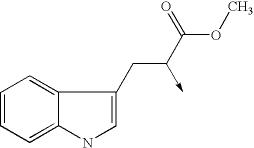

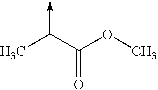

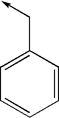

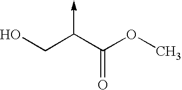

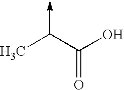

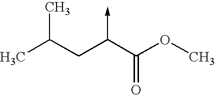

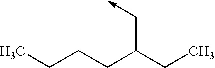

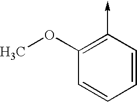

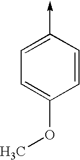

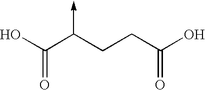

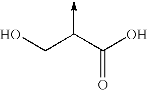

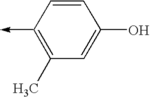

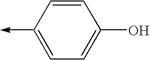

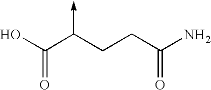

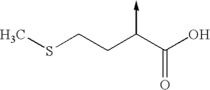

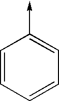

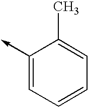

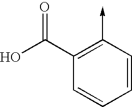

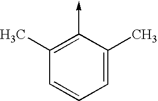

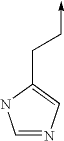

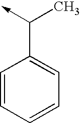

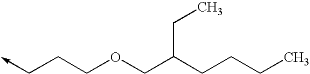

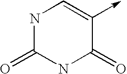

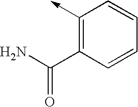

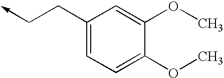

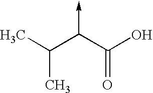

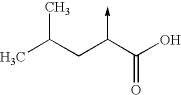

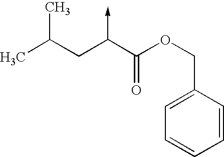

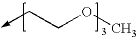

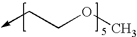

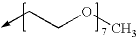

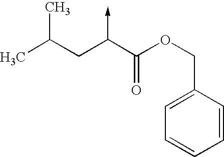

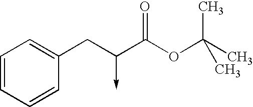

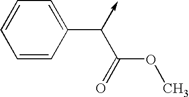

CH₂COOH
C₄H₉
C₅H₁₀OH
C₁₃H₂₇
C₁₆H₃₃

Of course, all combinations of said substituents R and X with said numbers for k, l, m and n are possible.

Table 2 summarizes the preferred individuals of the formula (I).

TABLE 2

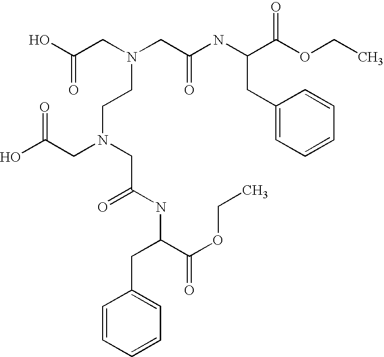

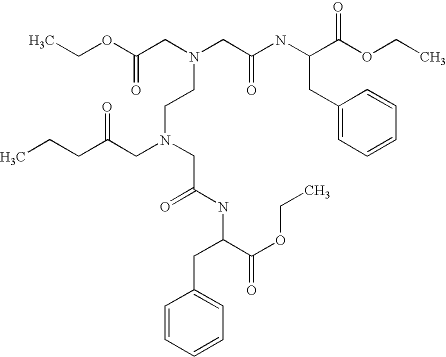

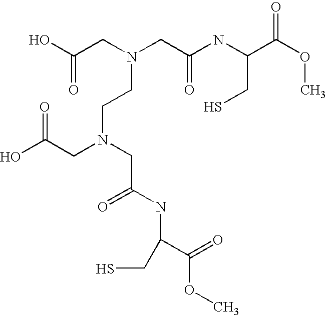

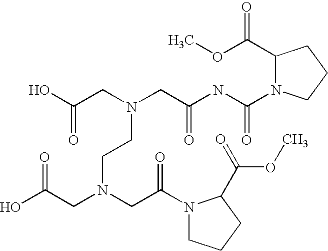

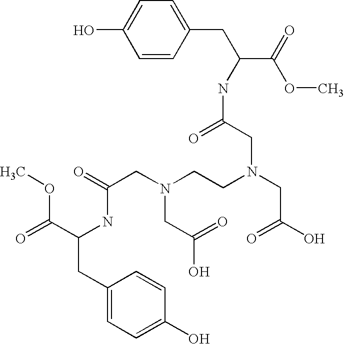

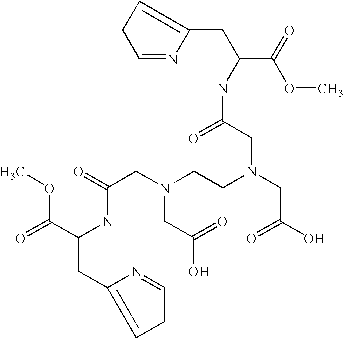

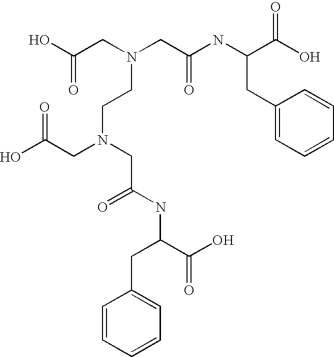

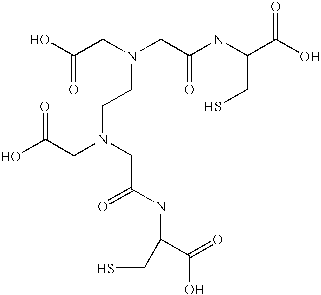

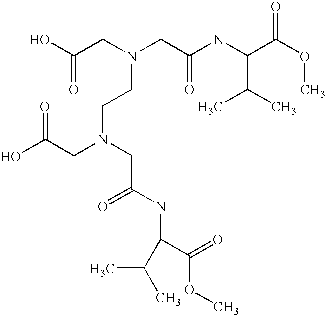

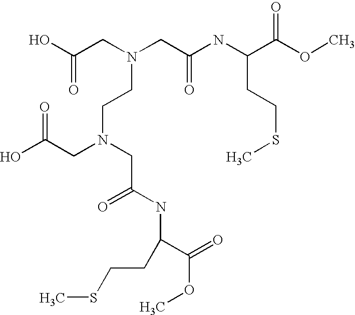

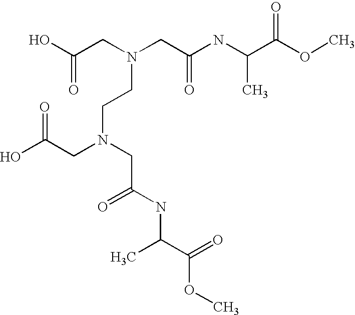

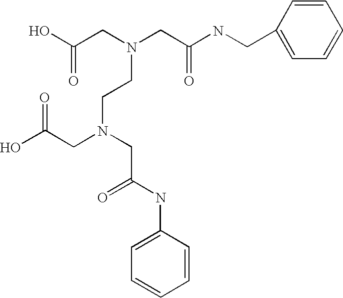

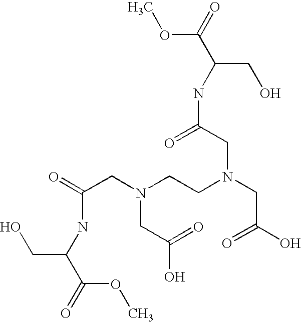

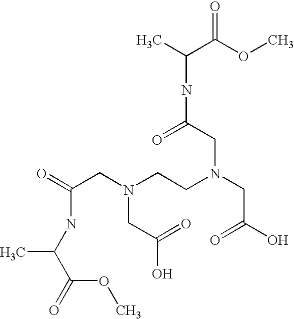

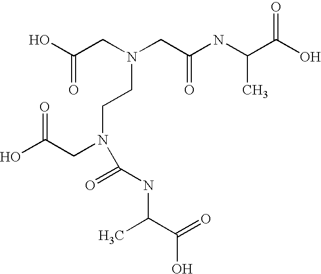

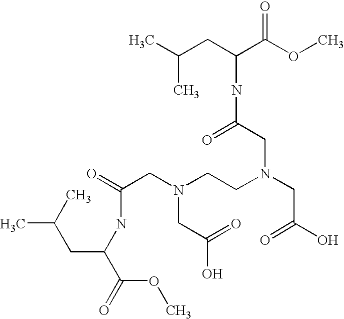

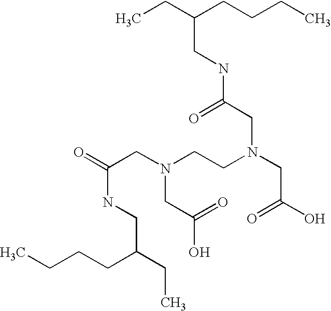

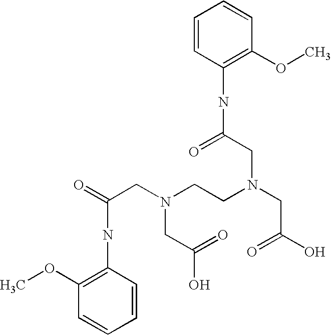

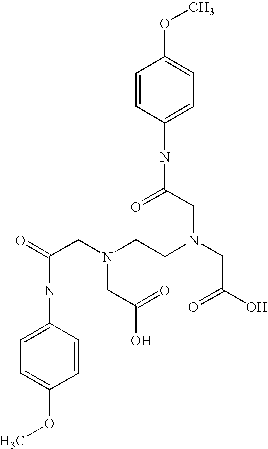

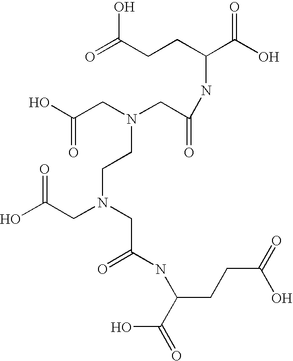

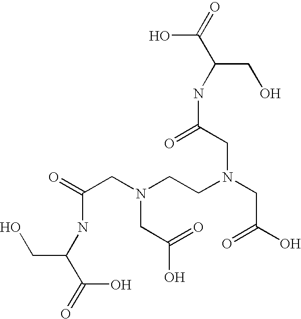

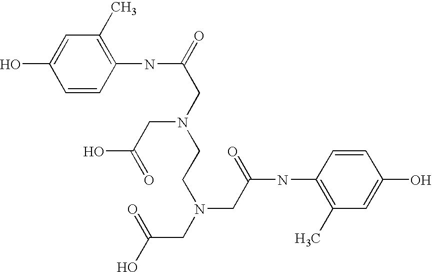

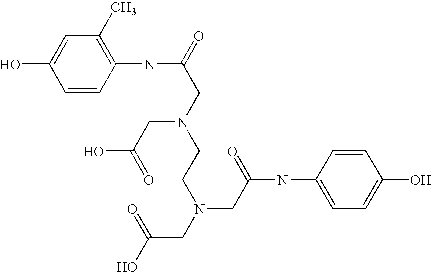

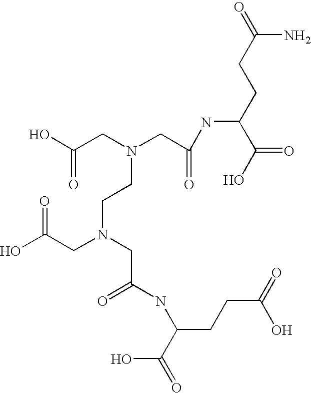

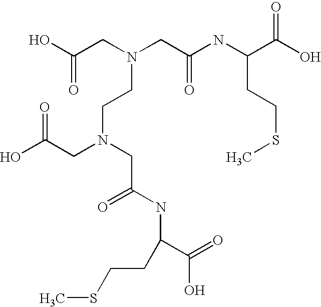

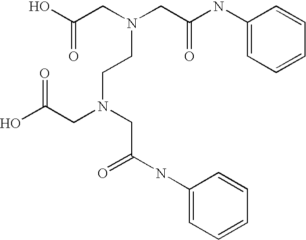

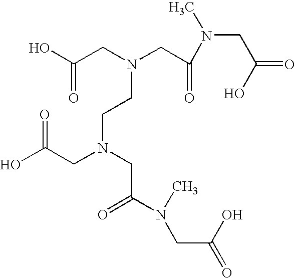

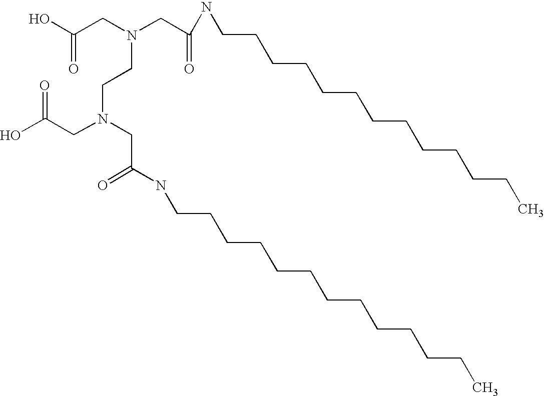

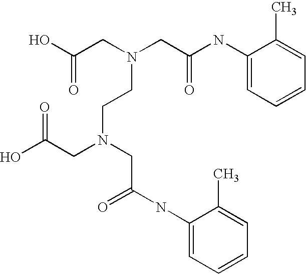

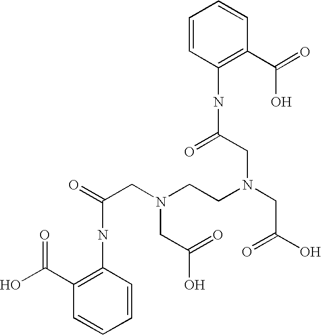

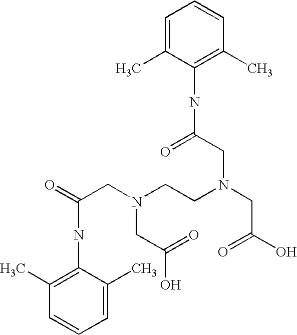

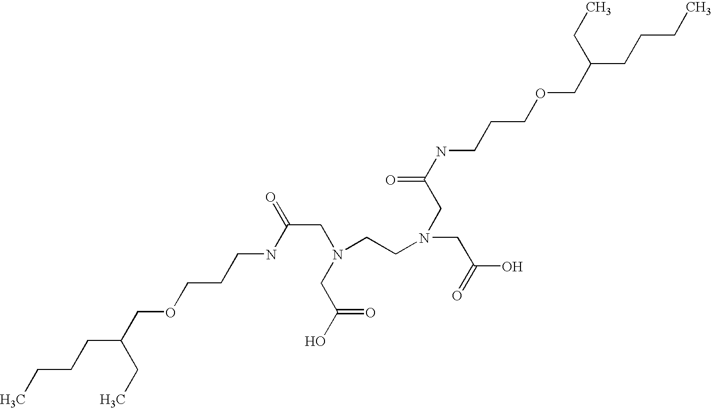

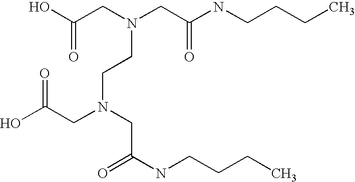

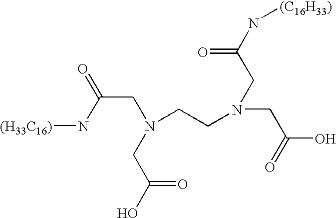

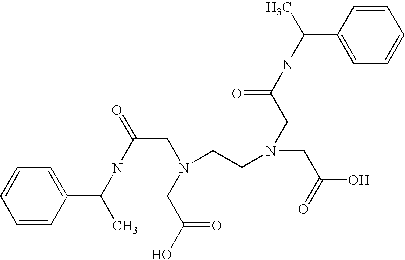

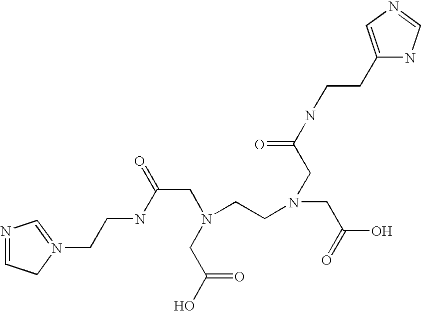

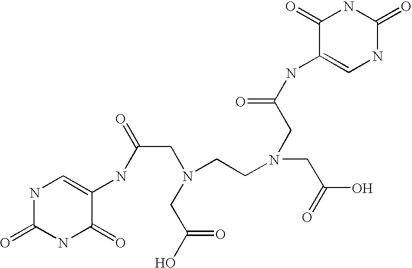

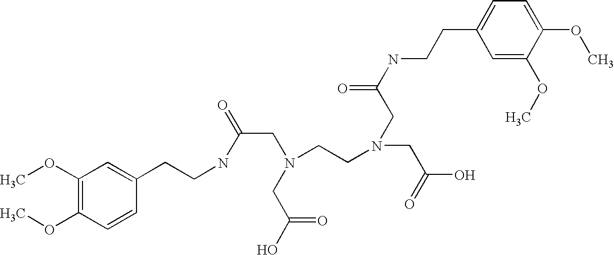

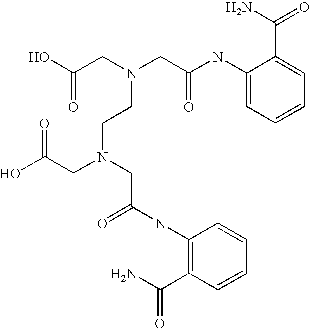

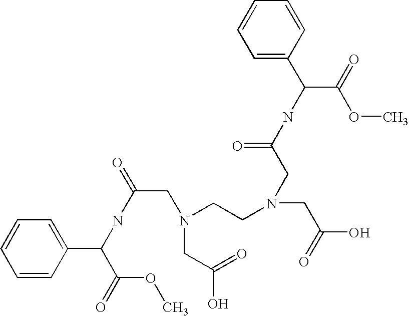

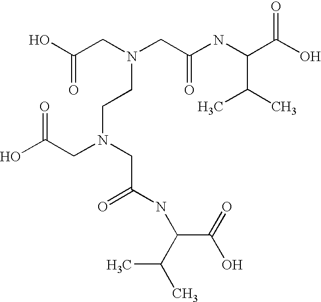

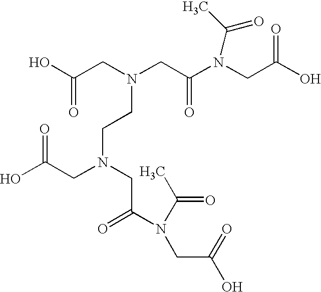

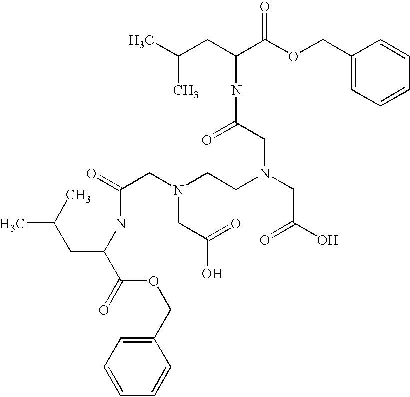

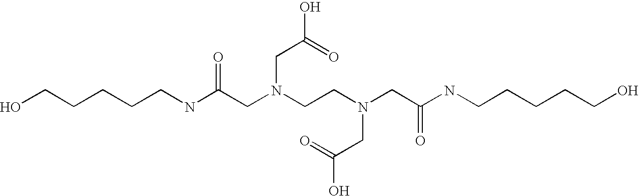

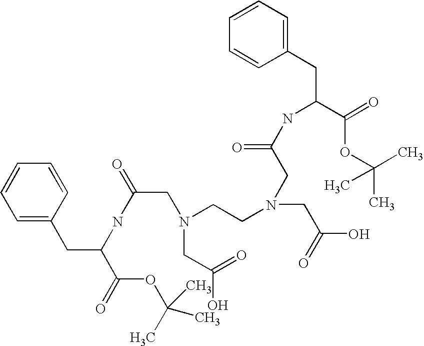

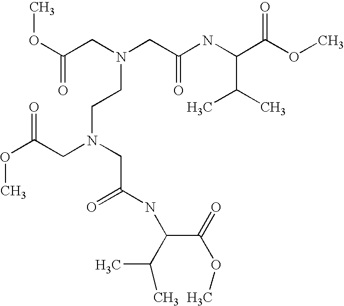

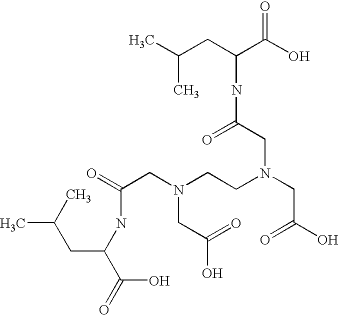

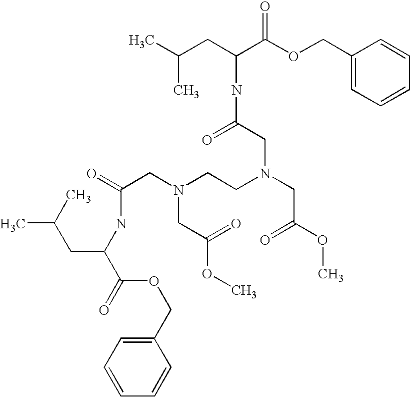

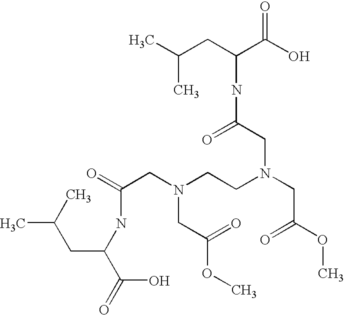

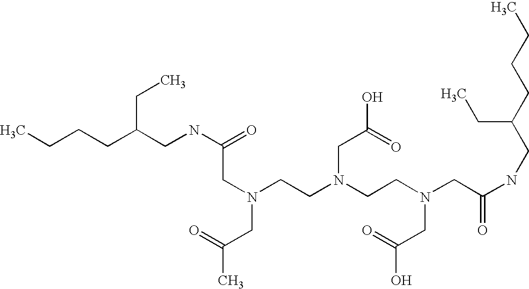

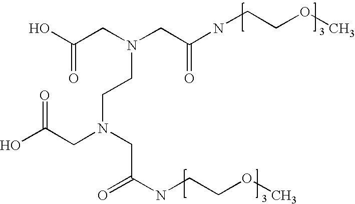

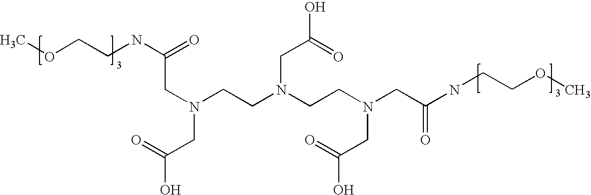

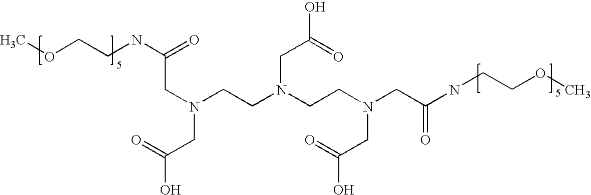

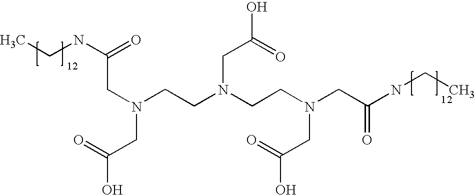

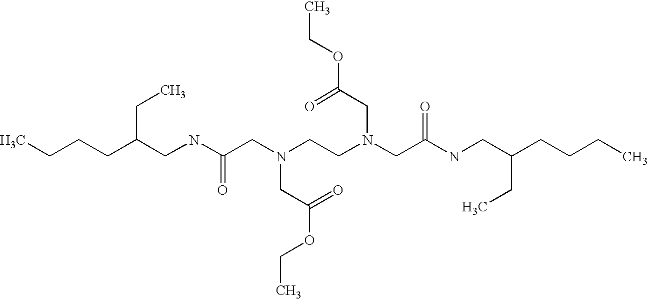

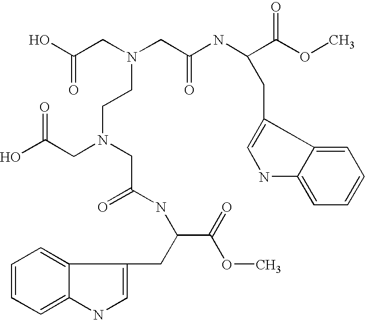

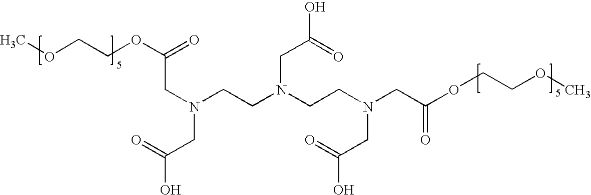

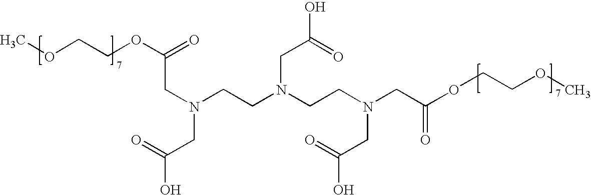

The preparation of these compounds is effected, for example, analogously to the synthesis routes described in DE-A 101 05 014 and CH-A 569 405. The compounds disclosed therein are used as metal complexes in metal deficiency phenomena or as contrast agents in diagnosis. A general synthesis route is disclosed in Bioorganic Medicinal Chemistry Letters 11 (2001), 2573.

Usually, the free radical scavengers are used individually or as a mixture, preferably not more than five, particularly preferably not more than four and very particularly preferably not more than three of the abovementioned free radical scavengers being used.

The amount in which the novel free radical scavengers are used in order to have a stabilizing effect on the polymerizable compound is determined in the course of experiments customary in the field.

For example, from 0.1 to 1 000 ppm, preferably from 1 to 900 ppm, particularly preferably from 10 to 800 ppm, very particularly preferably from 50 to 700 ppm and in particular from 100 to 500 ppm, based on the polymerizable compound, of a free radical scavenger or of a free radical scavenger mixture are used.

According to the invention, the free radical scavengers can advantageously be used together with at least one other compound known as a stabilizer and/or costabilizer. These are described, for example, in the prior German Patent Application with the Application Number 102 49 507.6 and in DE-A 102 58 329, DE-A 198 56 565 and EP-A 765 856.

Suitable costabilizers are oxygen-containing gases, phenolic compounds, quinones and hydroquinones, N-oxyl compounds, aromatic amines and phenylenediamines, imines, sulfonamides, oximes, hydroxylamines, urea derivatives, phosphorus-containing compounds, sulfur-containing compounds, complexing agents based on tetraazaannulene (TAA) and/or metal salts, and, if appropriate, mixtures thereof.

Oxygen-containing gases may be, for example, those gases which have an oxygen content of from 0.1 to 100, preferably from 0. 5 to 50, particularly preferably from 1 to 25, % by volume. For example, these may be nitrogen monoxide, nitrogen dioxide, oxygen or dinitrogen trioxide or air. These may be used individually, in any desired mixtures with one another or mixed with another gas, for example nitrogen, noble gases, steam, carbon monoxide, carbon dioxide or lower alkanes, preferably air or air/nitrogen mixtures.

Phenolic compounds are, for example, phenol, alkylphenols, for example o-, m- or p-cresol(methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol or 2,2′-methylenebis(6-tert-butyl-4-methylphenol), 4,4′-oxybiphenyl, 3,4-methylenedioxydiphenol (sesamol), 3,4-dimethylphenol, pyrocatechol (1,2-dihydroxybenzene), 2-(1′-methylcyclohex-1′-yl)-4,6-dimethylphenol, 2- or 4-(1′-phenyleth-1′-yl)phenol, 2-tert-butyl-6-methylphenol, 2,4,6-tris-tert-butylphenol, 2,6-di-tert-butylphenol, nonylphenol [CAS No. 1106649-2], octylphenol [CAS No.140-66-9], 2,6-dimethylphenol, bisphenol A, bisphenol B, bisphenol C, bisphenol F, bisphenol S, 3,3′,5,5′-tetrabromobisphenol A, 2,6-di-tert-butyl-p-cresol, Koresin® from BASF Aktiengesellschaft, methyl 3,5-di-tert-butyl-4-hydroxybenzoate, 4-tert-butylpyrocatechol, 2-hydroxybenzyl alcohol, 2-methoxy-4-methylphenol, 2,3,6-trimethylphenol, 2,4,5-trimethylphenol, 2,4,6-trimethylphenol, 2-isopropylphenol, 4-isopropylphenol, 6-isopropyl-m-cresol, n-octadecyl β-(3,5-di-tert-butyl-4-hydroxyphenyl)propionate, 1,1,3-tris(2-methyl-4-hydroxy-5-tert-butylphenyl)butane, 1,3,5-trimethyl-2,4,6-tris-(3,5-di-tert-butyl-4-hydroxybenzyl)benzene, 1,3,5,-tris(3,5-di-tert-butyl-4-hydroxybenzyl)isocyanurate, 1,3,5-tris(3,5-di-tert-butyl-4-hydroxyphenyl)propionyloxyethyl isocyanurate, 1,3,5-tris(2,6-dimethyl-3-hydroxy-4-tert-butylbenzyl)isocyanurate or pentaerythrityl tetrakis[p-(3,5-di-tert-butyl-4-hydroxyphenyl)propionate], 2,6-di-tert-butyl-4-dimethylaminomethylphenol, 6-sec-butyl-2,4-dinitrophenol, Irganox® 565, 1010, 1076, 1141, 1192, 1222 and 1425 from Ciba Spezialitätenchemie, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, 3-thia-1,5-pentanediol bis[(3′,5′-di-tert-butyl-4′-hydroxyphenyl)propionate], 4,8-dioxa-1,11-undecanediol bis[(3′,5′-di-tert-butyl-4′-hydroxyphenyl)propionate], 4,8-dioxa-1,11-undecanediol bis[(3′-tert-butyl-4′-hydroxy-5′-methylphenyl)propionate], 1,9-nonanediol bis[(3′,5′-di-tert-butyl-4′-hydroxyphenyl)propionate], 1,7-heptanediaminebis[3-(3′,5′-di-tert-butyl-4′-hydroxyphenyl)propionamide], 1,1-methanediaminebis[3-(3′,5′-di-tert-butyl-4′-hydroxyphenyl)propionamide], 3-(3′,5′-di-tert-butyl-4′-hydroxyphenyl)propionic acid hydrazide, 3-(3′,5′-dimethyl-4′-hydroxyphenyl)propionic acid hydrazide, bis(3-tert-butyl-5-ethyl-2-hydroxyphen-1-yl)methane, bis(3,5-di-tert-butyl-4-hydroxyphen-1-yl)methane, bis[3-(1′-methylcyclohex-1′-yl)-5-methyl-2-hydroxyphen-1-yl]methane, bis(3-tert-butyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris-[1′-(3Δ,5″-di-tert-butyl-4″-hydroxyphen-1″-yl)meth-1′-yl]-2,4,6-trimethylbenzene, 1,1,4-tris(5′-tert-butyl-4′-hydroxy-2′-methylphen-1′-yl)butane and tert-butyleatechol, and aminophenols, such as p-aminophenol, nitrosophenols, such as p-nitrosophenol, p-nitroso-o-cresol, alkoxyphenols, for example methoxyphenol (guajacol, pyrocatechol monomethyl ether), 2-ethoxyphenol, 2-isopropoxyphenol, 4-methoxyphenol (hydroquinone monomethyl ether), mono- or di-tert-butyl-4-methoxyphenol, 3,5-di-tert-butyl-4-hydroxyanisole, 3-hydroxy-4-methoxybenzyl alcohol, 2,5-dimethoxy-4-hydroxybenzyl alcohol (syringa alcohol), 4-hydroxy-3-methoxybenzaldehyde (vanillin), 4-hydroxy-3-ethoxybenzaldehyde (ethylvanillin), 3-hydroxy-4-methoxybenzaldehyde (isovanillin), 1-(4-hydroxy-3-methoxyphenyl)ethanone (acetovanillone), eugenol, dihydroeugenol, isoeugenol, tocopherols, such as α-, β-, γ-, δ- and ε-tocopherol, tocol, α-tocopherolhydroquinone, and 2,3-dihydro-2,2-dimethyl-7-hydroxybenzofuran (2,2-dimethyl-7-hydroxycoumaran).

Suitable quinones and hydroquinones are, for example hydroquinone or hydroquinone monomethyl ether(4-methoxyphenol), methylhydroquinone, 2,5-di-tert-butylhydroquinone, 2-methyl-p-hydroquinone, 2,3-dimethylhydroquinone, trimethylhydroquinone, 4-methylpyrocatechol, tert-butylhydroquinone, 3-methylpyrocatechol, benzoquinone, 2-methyl-p-hydroquinone, 2,3-dimethylhydroquinone, tdmethylhydroquinone, tert-butylhydroquinone, 4-ethoxyphenol, 4-butoxyphenol, hydroquinone monobenzyl ether, p-phenoxyphenol, 2-methylhydroquinone, tetramethyl-p-benzoquinone, diethyl-1,4-cyclohexanedion 2,5-dicarboxylate, phenyl-p-benzoquinone, 2,5-dimethyl-3-benzyl-p-benzoquinone, 2-isopropyl-5-methyl-p-benzoquinone (thymoquinone), 2,6-diisopropyl-p-benzoquinone, 2,5-dimethyl-3-hydroxy-p-benzoquinone, 2,5-dihydroxy-p-benzoquinone, embelin, tetrahydroxy-p-benzoquinone, 2,5-dimethoxy-1,4-benzoquinone, 2-amino-5-methyl-p-benzoquinone, 2,5-bisphenylamino-1,4-benzoquinone, 5,8-dihydroxy-1,4-naphthoquinone, 2-anilino-1,4-naphthoquinone, anthraquinone, N,N-dimethylindoaniline, N,N-diphenyl-p-benzoquinonediimine, 1,4-benzoquinone dioxime, coerulignone, 3,3′-di-tert-butyl-5,5′-dimethyldiphenoquinone, p-rosolic acid (aurin), 2,6-di-tert-butyl-4-benzylidenebenzoquinone and 2,5-di-tert-amylhydroquinone.

Suitable N-oxyls (nitroxyl or N-oxyl radicals, compounds which have at least one >N—O● group) are, for example, 4-hydroxy-2,2,6,6-tetramethylpiperidin-N-oxyl, 4-oxo-2,2,6,6-tetramethylpiperidin-N-oxyl, 4-methoxy-2,2,6,6-tetramethylpiperidin-N-oxyl, 4-acetoxy-2,2,6,6-tetramethylpiperidin-N-oxyl, 2,2,6,6-tetramethylpiperidin-N-oxyl, Uvinul® 4040P from BASF Aktiengesellschaft, 4,4′,4″-tris(2,2,6,6-tetramethylpiperidin-N-oxyl)phosphite, 3-oxo-2,2,5,5-tetramethylpyrrolidin-N-oxyl, 1-oxyl-2,2,6,6-tetramethyl-4-methoxypiperidine, 1-oxyl-2,2,6,6-tetramethyl-4-trimethylsilyloxypiperidine, 1-oxyl-2,2,6,6-tetramethylpiperidin-4-yl 2-ethylhexanoate, 1-oxyl-2,2,6,6-tetramethylpiperidin-4-yl sebacate, 1-oxyl-2,2,6,6-tetramethylpiperidin-4-yl stearate, 1-oxyl-2,2,6,6-tetramethylpiperidin-4-yl-benzoate, 1-oxyl-2,2,6,6-tetramethylpiperidin-4-yl (4-tert-butyl)benzoate, bis(1-oxyl-2,2,6,6-tetramethylpiperidin4-yl) succinate, bis(1-oxyl-2,2,6,6-tetramethylpiperidin-4-yl) adipate, bis(1-oxyl-2,2,6,6-tetramethylpiperidin-4-yl)1,10-decanedioate, bis(1-oxyl-2,2,6,6-tetramethylpiperidin4-yl)n-butylmalonate, bis(1-oxyl-2,2,6,6-tetramethylpiperidin-4-yl) phthalate, bis(1-oxyl-2,2,6,6-tetramethylpiperidin-4-yl)isophthalate, bis(1-oxyl-2,2,6,6-tetramethylpiperidin4-yl) terephthalate, bis(1-oxyl-2,2,6,6-tetramethylpiperidin-4-yl) hexahydroterephthalate, N,N′-bis(1-oxyl-2,2,6,6-tetramethylpiperidin-4-yl)adipamide, N-(1-oxyl-2,2,6,6-tetramethylpiperidin-4-yl)caprolactam, N-(1-oxyl-2,2,6,6-tetramethylpiperidin-4-yl)dodecylsuccinimide, 2,4,6-tris[N-butyl-N-(1-oxyl-2,2,6,6-tetramethylpiperidin-4-yl]triazine, N,N′-bis(1-oxyl-2,2,6,6-tetramethylpiperidin-4-yl)-N,N′-bisformyl-1,6-diaminohexane and 4,4′-ethylenebis(1-oxyl-2,2,6,6-tetramethylpiperazin-3-one).

Suitable aromatic amines or phenylenediamines are, for example, N,N-diphenylamine, N-nitrosophenylamine, nitrosodiethylaniline, p-phenylenediamine, N,N′-dialkyl-p-phenylenediamine. it being possible for the alkyl radicals to be identical or different and, in each case independently of one another, to comprise 1 to 4 carbon atoms and to be straight-chain or branched, for example N,N′-diisobutyl-p-phenylenediamine, N,N′-diisopropyl-p-phenylenediamine, Irganox® 5057 from Ciba Spezialitätenchemie, N-phenyl-p-phenylenediamine, N,N′-diphenyl-p-phenylenediamine, N-isopropyl-N-phenyl-p-phenylenediamine, N,N′-di-sec.-butyl-p-phenylenediamine (Kerobit® BPD from BASF Aktiengesellschaft), N-phenyl-N′-isopropyl-p-phenylenediamine (Vulkanox® 4010 from Bayer AG), N-(1,3-dimethylbutyl)-N′-phenyl-p-phenylenediamine, N-phenyl-2-naphthylamine, iminodibenzyl, N,N′-diphenylbenzidine, N-phenyltetraaniline, acridone, 3-hydroxydiphenylamine and 4-hydroxydiphenylamine.

Imines are, for example, methylethylimine, (2-hydroxyphenyl)benzoquinonimine, (2-hydroxyphenyl)benzophenonimine, N,N-dimethylindoaniline, thionine (7-amino-3-imino-3H-phenothiazine) and methylene violet (7-dimethylamino-3-phenothiazinone).

Sulfonamides effective as a stabilizer are, for example, N-methyl-4-toluenesulfonamide, N-tert-butyl-4-toluenesulfonamide, N-tert-butyl-N-oxyl-4-toluenesulfonamide, N,N′-bis(4-sulfanilamide)piperidine, 3-{[5-(4-aminobenzoyl)-2,4-dimethylbenzenesulfonyl]ethylamino}-4-methylbenzenesulfonic acid, as in DE-A 102 58 329.

Oximes may be, for example, aldoximes, ketoximes or amidoximes, as described, for example, in DE-A 101 39 767, preferably diethyl ketoxime, acetone oxime, methyl ethyl ketoxime, cylcohexanone oxime, benzaldehyde oxime, benzil dioxime, dimethylglyoxime, 2-pyridinaldoxime, salicylaldoxime, phenyl-2-pyridyl ketoxime, 1,4-benzoquinone dioxime, 2,3-butandione dioxime, 2,3-butandione monooxime, 9-fluorenone oxime, 4-tert-butyl-cyclohexanone oxime, ethyl N-ethoxy-acetimidate, 2,4-dimethyl-3-pentanone oxime, cyclododecanone oxime, 4-heptanone oxime and di-2-furanylethanedione dioxime or other aliphatic or aromatic oximes or the reaction products thereof with alkyl transfer reagents, such as alkyl halides, triflates, sulfonates, tosylates, carbonates, sulfates, phosphates or the like.

Hydroxylamines are, for example, N,N-diethylhydroxylamine and those which are disclosed in the International Application with the Application Number PCT/EP/03/03139.

Suitable urea derivatives are, for example, urea or thiourea.

Phosphorus-containing compounds are, for example, triphenylphosphine, triphenyl phosphite, hypophosphorous acid, trinonyl phosphite, triethyl phosphite or diphenylisopropylphosphine.

Suitable sulfur-containing compounds are, for example, diphenyl sulfide, phenothiazine and sulfur-containing natural substances, such as cysteine.

Complexing agents based on tetraazaannulene (TAA) are, for example, dibenzotetraaza[14]annulenes and porphyrins, as mentioned in Chem. Soc. Rev. 27 (1998), 105-115.

Metal salts are, for example, copper, manganese, cerium, nickel and chromium carbonate, chloride, dithiocarbamate, sulfate, salicylate, acetate, stearate or ethylhexanoate.

Preferred costabilizers are oxygen-containing gases, phenothiazine, o-, m- or p-cresol (methylphenol), 2-tert-butyl-4-methylphenol, 2,6-di-tert-butyl-4-methylphenol, 2-tert-butylphenol, 4-tert-butylphenol, 2,4-di-tert-butylphenol, pyrocatechol (1,2-Dihydroxybenzene), 2,6-di-tert-butylphenol, 4-tert-butyl-2,6-dimethylphenol, octylphenol [140-66-9], nonylphenol [11066-49-2], 2,6-dimethylphenol, 2,6-di-tert-butyl-p-cresol, bisphenol A, tert-butylcatechol, hydroquinone, hydroquinone monomethyl ether or methylhydroquinone, 2,2,6,6-tetramethylpiperidin-N-oxyl, 4-hydroxy-2,2,6,6-tetramethylpiperidin-N-oxyl, 4-oxo-2,2,6,6-tetramethylpiperidin-N-oxyl and manganese (II) acetate, cerium(III) carbonate, cerium(III) acetate or cerium(III) ethylhexanoate, cerium(III) stearate and mixtures thereof of different composition.

Air, air/nitrogen mixtures, phenothiazine, o-, m- or p-cresol (methylphenol), 2,6-di-tert-butyl-4-methylphenol, 4-tert-butylphenol, 4-tert-butyl-2,6-dimethylphenol, octylphenol [140-66-9], nonylphenol [11066-49-2], 2,6-dimethylphenol, 2,6-di-tert-butyl-p-cresol, tert-butylcatechol, hydroquinone, hydroquinone monomethyl ether or methylhydroquinone, 2,2,6,6-tetramethylpiperidin-N-oxyl, 4-hydroxy-2,2,6,6-tetramethylpiperidin-N-oxyl, 4-oxo-2,2,6,6-tetramethylpiperidin-N-oxyl and cerium(III) acetate or cerium(III) ethylhexanoate and mixtures of at least two of said components are particularly suitable.

Air, air/nitrogen mixtures, phenothiazine, hydroquinone and hydroquinone monomethyl ether and 2,2,6,6-tetramethylpiperidin-N-oxyl, 4-hydroxy-2,2,6,6-tetramethylpiperidin-N-oxyl, 4-oxo-2,2,6,6-tetramethylpiperidin-N-oxyl, N,N′-di-sec-butyl-p-phenylendiamine, cerium(III) acetate or cerium(III) ethylhexanoate and mixtures thereof are particularly preferred.

The method of addition of the novel stabilizer and of any costabilizers to be used is not restricted. The added novel stabilizer can be added in each case individually or as a mixture with further novel stabilizers and/or with abovementioned costabilizers, in liquid or in dissolved form in a suitable solvent, it being possible for this solvent itself to be a stabilizer, as described, for example, in DE-A 102 00 583. Suitable solvents are furthermore, for example, material streams from the preparation of the polymerizable compound. These may be, for example, the pure products, i.e. the polymerizable compounds, in a purity of, as a rule, 95% or more, preferably 98% or more and particularly preferably 99% or more, but also the starting materials used for the preparation of the polymerizable compounds, in a purity of 95% or more, preferably 98% or more and particularly preferably 99% or more, or those material streams which contain the starting materials and/or products and/or intermediates and/or byproducts.

The concentration of the solutions used is limited only by the solubility of the stabilizer/stabilizer mixture in the solvent; for example, it may be 0.1-50, preferably 0.2-25, particularly preferably 0.3-10, very particularly preferably 0.5-5, % by weight.

Of course, the novel free radical scavengers or free radical scavenger mixtures may also be used in the form of a melt, for example if the melting point is below 120° C., preferably below 100° C., particularly preferably below 80° C. and in particular below 60° C.

In a further preferred embodiment, the novel free radical scavengers or free radical scavenger mixtures are used in the form of a melt with a phenol having a melting point below 120° C., preferably below 100° C., particularly preferably below 80° C. and in particular below 60° C. as a costabilizer. The phenol is particularly preferably selected from p-aminophenol, p-nitrosophenol, 2-tert-butylphenol, 4-tert-butylphenol, 2,4-di-tert-butylphenol, 2-methyl-4-tert-butylphenol, 4-tert-butyl-2,6-dimethylphenol, hydroquinone and hydroquinone monomethyl ether.

As described above, from 0.1 to 1 000 ppm, based on the polymerizable compound, of the novel free radical scavenger or of a mixture of free radical scavengers are used. If a mixture of novel free radical scavengers is used with costabilizers, from 0.1 to 5 000 ppm, preferably from 1 to 4 000 ppm, particularly preferably from 5 to 2 500 ppm, particularly preferably from 10 to 1 000 ppm and in particular from 50 to 750 ppm, based on the polymerizable compound, are used.

If a mixture of a plurality of stabilizers or costabilizers is used, these may both be fed in independently of one another at different metering points or at the same metering point and, independently of one another, dissolved in different solvents.

According to the invention, the stabilizers/stabilizer mixtures are preferably used at those points for the polymerizable compound, if exposed to a risk of polymerization, for example due to high purity, a long residence time and/or a high temperature.

These may be, for example, absorption units, desorption units, rectification units, for example distillation apparatuses or rectification columns, evaporators, for example natural circulation or forced circulation evaporators, condensers or vacuum units.

For example, the stabilizers can be metered in at the top of a rectification unit, for example sprayed or atomized into the top of the rectification unit or take-off internals or via the internals having separation activity, e.g. trays, packings, baffles or beds, or metered, for example sprayed, together with the reflux into a condenser, so that the top of the condenser and/or the cooling surfaces are wet, or into a vacuum unit, as described in EP-A 1 057 804, or as sealing liquid into a liquid ring pump, as described in DE-A 101 43 565.

The free radical scavengers to be used according to the invention may be used both as storage stabilizers and as transport stabilizers, i.e. for stabilizing the pure polymerizable compounds.

The present invention furthermore relates to stabilizer mixtures comprising
i) at least one free radical scavenger which contains at least two glycine units, with the proviso that the free radical scavenger is not selected from ethylenediaminetetraacetic acid (EDTA), diethylenetriaminepentaacetic acid (DTPA), trans-1,2-cyclohexanediaminetetraacetic acid (CYDTA) and the alkali metal and alkaline earth metal salts thereof and
ii) at least one further stabilizer or costabilizer.

Therein, free radical scavengers having at least two glycine units i) are compounds which preferably contain at least one amide and/or ester unit, particularly preferably free radical scavengers of the formula (I). Of course, the present invention also relates to stabilizer mixtures comprising the abovementioned preferred novel free radical scavengers.

Combinations of all abovementioned components are possible.

Stabilizers or costabilizers are the abovementioned oxygen-containing gases, phenolic compounds, quinones and hydroquinones, N-oxyl compounds, aromatic amines and phenylenediamines, imines, sulfonamides, oximes, hydroxylamines, urea derivatives, phosphorus-containing and/or sulfur-containing compounds, TAA-based complexing agents and/or metal salts.

Stabilizer mixtures comprising the free radical scavenger and phenothiazine, free radical scavenger/hydroquinone, free radical scavenger/hydroquinone monomethyl ether, free radical scavenger/4-hydroxy-2,2,6,6-tetramethylpiperidin-N-oxyl, free radical scavenger/4-oxo-2,2,6,6-tetramethylpiperidin-N-oxyl, free radical scavenger/2,2,6,6-tetramethylpiperidin-N-oxyl, free radical scavenger/phenothiazine/hydroquinone monomethyl ether, free radical scavenger/phenothiazine/4-hydroxy-2,2,6,6-tetramethylpiperidin-N-oxyl or free radical scavenger/hydroquinone monomethyl ether/4-hydroxy-2,2,6,6-tetramethylpiperidin-N-oxyl and, if appropriate, in each case at least one of said cerium salts, in each case in the presence or absence of an oxygen-containing gas, preferably in the presence thereof, are preferred.

The novel stabilizer mixtures contain the components i) and ii) in weight ratios i): ii) of from 1:100 to 100:1, preferably from 1:50 to 50:1, particularly preferably from 1:10 to 10:1 and in particular from 1:5 to 5:1.

The present invention furthermore relates to mixtures which contain the abovementioned novel stabilizer mixtures and at least one polymerizable compound. All combinations of novel stabilizer mixtures with polymerizable compounds are possible.

The present invention also relates to the use of mixtures which contain the abovementioned stabilizer mixtures for stabilizing polymerizable compounds to polymerization during working-up, storage and/or transport.

In the context of the present invention, polymerizable compounds are those having at least one ethylenically unsaturated group. These are selected from mono-, di- or triethylenically unsaturated C₃-C₈-carboxylic acids, C₁-C₂₀-esters, C₁-C₂₀-amides, C₁-C₂₀-nitriles and C₁-C₂₀-anhydrides of these mono-, di- or triethylenically unsaturated C₃-C₈-carboxylic acids, vinyl esters of carboxylic acids of up to 20 carbon atoms, vinyl ethers of alcohols of 1 to 10 carbon atoms, vinylaromatics and vinylheteroaromatics of up to 20 carbon atoms, vinyllactams having 3 to 10 carbon atoms in the ring, open-chain N-vinylamide compounds and N-vinylamine compounds, vinyl halides, aliphatic optionally halogenated hydrocarbons having 2 to 8 carbon atoms and 1 or 2 double bonds, vinylidenes or mixtures of these monomers.

Preferred mono-, di- or triethylenically unsaturated C₃-C₆-carboxylic acids are, for example, (meth)acrylic acid, dimethacrylic acid, ethacrylic acid, citraconic acid, methylenemalonic acid, crotonic acid, fumaric acid, mesaconic acid, itaconic acid, maleic acid and the C₁-C₂₀-alkyl esters, C₁-C₂₀-amides, C₁-C₂₀-nitriles, C₁-C₂₀-aldehydes and C₁-C₂₀-anhydrides thereof, e.g. methyl(meth)acrylate, ethyl(meth)acrylate, n-butyl(meth)acrylate, n-propyl(meth)acrylate, isopropyl(meth)acrylate, 2-ethylhexyl(meth)acrylate, aryl(meth)acrylate, monomethyl maleate, dimethyl maleate, monoethyl maleate, diethyl maleate, alkylene glycol(meth)acrylates, (meth)acrylamide, N-dimethyl(meth)acrylamide, N-tert-butyl(meth)acrylamide, (meth)acrylonitrile, (meth)acrolein, (meth)acrylic anhydride, itaconic anhydride, maleic anhydride and the monoesters thereof. Cationic monomers of this group are, for example, dialkylaminoalkyl(meth)acrylates and dialkylaminoalkyl(meth)acrylamides, such as dimethylaminomethyl(meth)acrylate, dimethylaminoethyl(meth)acrylate, diethylaminoethyl(meth)acrylate and the salts of the last-mentioned monomers with carboxylic acids or mineral acids and the quaternized products.

Further monomers of this group are, for example, also hydroxyl-containing monomers, in particular C₁-C₁₀-hydroxyalkyl(meth)acrylates, such as hydroxyethyl(meth)acrylate, hydroxypropyl(meth)acrylate, hydroxybutyl(meth)acrylate and hydroxyisobutyl(meth)acrylate.

Further monomers of this group are phenoxyethyl glycol mono(meth)acrylate, glycidyl(meth)acrylate, trimethylolpropane triacrylate, ureidomethyl methacrylate, amino(meth)acrylates, such as 2-aminoethyl(meth)acrylate.

Vinyl esters of carboxylic acids of 1 to 2 carbon atoms are, for example, vinyl laurate, vinyl stearate, vinyl propionate, vinyl versatate and vinyl acetate.

Examples of vinyl ethers of alcohols of 1 to 10 carbon atoms are methyl vinyl ether, ethyl vinyl ether, butyl vinyl ether, 4-hydroxybutyl vinyl ether, vinyl isobutyl ether and dodecyl vinyl ether.

Examples of suitable vinylaromatic and vinylheteroaromatic compounds are vinyltoluene, α- and p-methylstyrene, α-butylstyrene, 4-n-butylstyrene, 4-n-decylstyrene, styrene, divinylbenzene, 2-vinylpyridine, N-vinylimidazole, N-vinylpiperidone, N-vinyl-2-methylimidazole and N-vinyl-4-methylimidazole.

Vinyllactams having 3 to 10 carbon atoms in the ring are, for example, N-vinylcaprolactam, N-vinylpyrrolidone, laurolactam, oxygenated purines, such as xanthine or derivatives thereof, such as 3-methylxanthine, hypoxanthine, guanine, theophylline, caffeine, adenine or theobromine.

Furthermore, open-chain N-vinylamide compounds and N-vinylamine compounds, for example N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide and N-vinyl-N-dimethylamine, N-vinyl-N-methylethylamine, N-vinyl-N-diethylamine can be stabilized by the novel process.

The vinyl halides are ethylenically unsaturated compounds substituted by chlorine, fluorine or bromine, for example vinyl chloride, vinyl fluoride and vinylidene chloride.

Examples of aliphatic optionally halogenated hydrocarbons having 2 to 8 carbon atoms and 1 or 2 olefinic double bonds are ethylene, propylene, 1-butene, 2-butene, isobutene, butadiene, isoprene and chloroprene.

An example of vinylidenes is vinylidene cyanide.

Further polymerizable compounds are N-vinylcaprolactam, vinylphosphoric acids, vinylacetic acid, allylacetic acid, N-vinylcarbazole, hydroxymethyl vinyl ketone, N,N-divinylethyleneurea, vinylene carbonate, tetrafluoroethylene, hexafluoropropene, nitroethylene, α-chloroacrylate, α-cyanoacrylate, methylenemalonate, α6-cyanosorbate, cyclopentadiene, cylcopentene, cyclohexene and cyclododecene.

In a preferred embodiment of the novel process, the free radical scavengers are used for stabilizing mono-, di- or triethylenically unsaturated C₃-C₈-carboxylic acids and the C₁-C₂₀-alkyl esters thereof or N-vinylcaprolactam, N-vinylformamide, N-vinylimidazole, N-vinylpyrrolidone, vinylphosphoric acids, N-vinylcarbazole, N,N-divinylethyleneurea, trimethylolpropane acrylate, ureidomethyl methacrylate, styrene, butadiene or isoprene.

Preferred unsaturated C₃-C₈-carboxylic acids are, for example, acrylic acid and methacrylic acid and the C₁-C₈-alkyl esters thereof, e.g. methyl(meth)acrylate, ethyl(meth)acrylate, n-butyl(meth)acrylate and 2-ethylhexyl(meth)acrylate.

In a preferred embodiment, the novel free radical scavengers are used in a process as described in DE-A 100 64 642. For this purpose, in a process for working up (meth)acrylic acid in the presence of at least one stabilizer, a stabilizer-containing mixture originating from the working-up and substantially freed from (meth)acrylic acid is fed to a distillation apparatus, and a stabilizer-containing low-boiler stream obtained from said apparatus is recycled to the working-up.

Novel free radical scavengers particularly suitable for such a process are those whose vapor pressures at 141° C. (boiling point of acrylic acid) at atmospheric pressure are at least 15, preferably from 20 to 800, particularly preferably from 25 to 500, very particularly preferably from 25 to 250, in particular from 25 to 160, hPa, and mixtures thereof.

In a further preferred embodiment, the novel free radical scavengers are used in a process for working up N-vinyl monomers, such as said vinyl esters, vinyl ethers, vinylaromatics and vinylheteroaromatics, and the open-chain N-vinylamide compounds and N-vinylamine compounds.

Of course, the field of use of the novel process also relates to storage and transport of these polymerizable compounds.

ppm and percentage data used in this document are by weight, unless stated otherwise.

The examples which follow illustrate the invention but do not restrict it to these examples.

### EXAMPLE 1

0.5 ml of freshly thawed acrylic acid doubly distilled to remove the storage stabilizer was filled into 1.8 ml ampoules under an air atmosphere.

The samples were stored in a through-circulation dryer at 120° C.

In each test series, 3 ampoules were filled with each acrylic acid sample and were tested, and the mean value of the time taken for complete polymerization was visually assessed.

The average standard deviation within a test series was about 2-4%.

The concentrations were 25 ppm of free radical scavenger plus 10 ppm of phenothiazine (PTZ), unless stated otherwise.

The relative efficiency is calculated from the quotient of the time taken for polymerization of the sample of free radical scavenger and PTZ and the time taken for polymerization of the reference sample. The reference sample used was pure PTZ, and the relative efficiency of the reference sample was accordingly 1.0.

The results are summarized in table 3.

TABLE 3
4.76

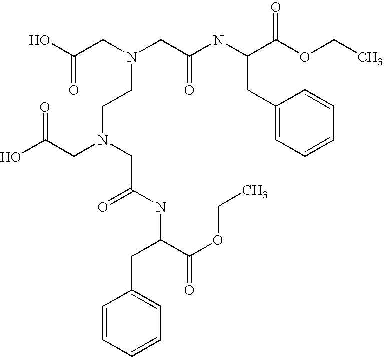

4.30

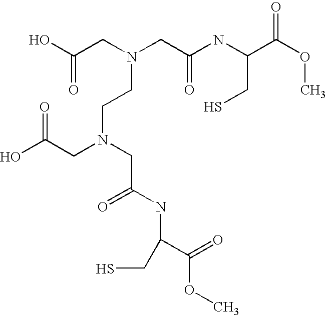

6.01

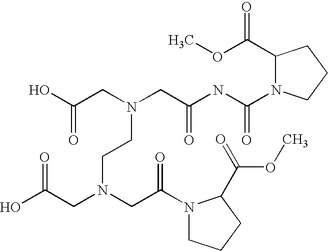

7.55

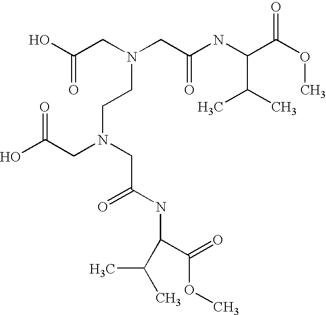

5.30

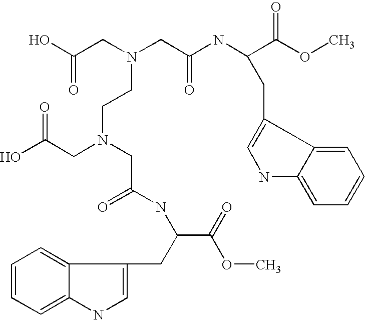

4.06

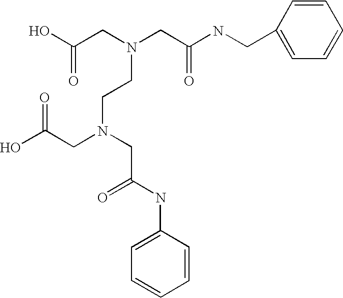

6.50

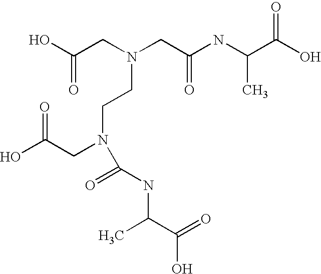

7.30

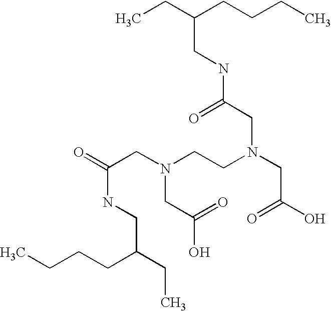

7.00

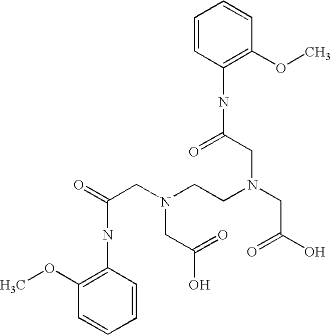

2.70

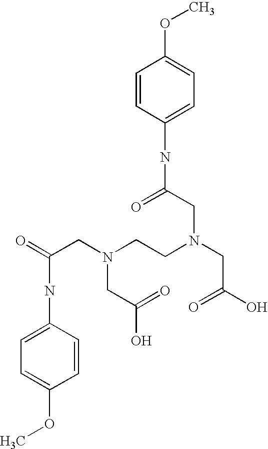

6.54

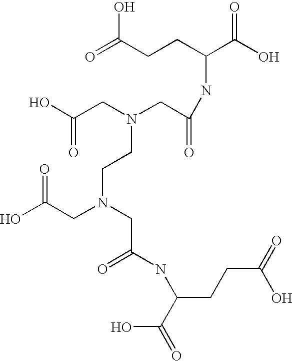

6.66

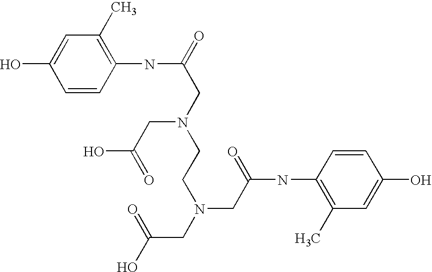

11.42

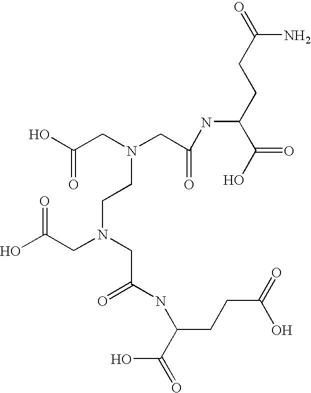

9.59

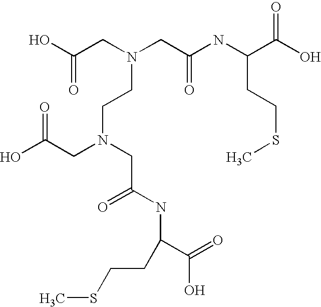

8.09

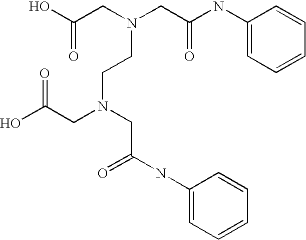

8.73

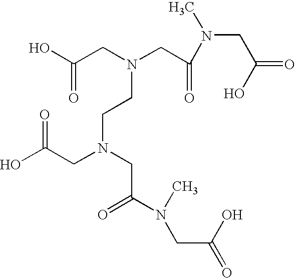

3.22

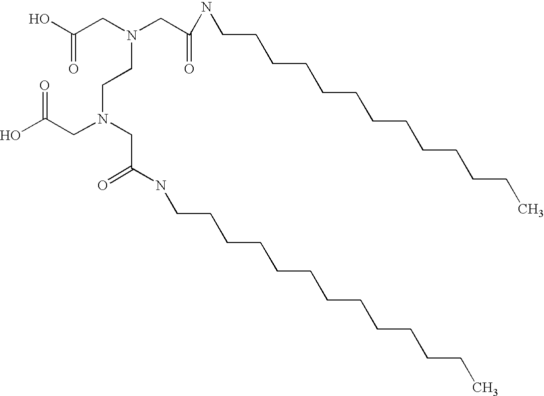

6.42

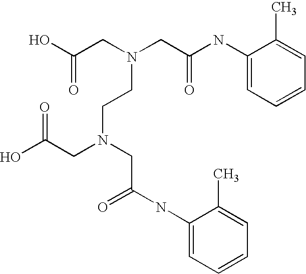

5.47

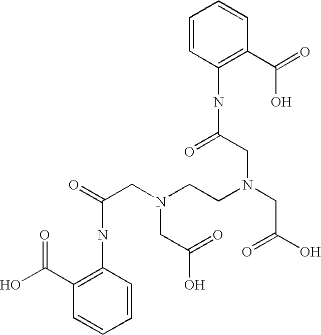

3.78

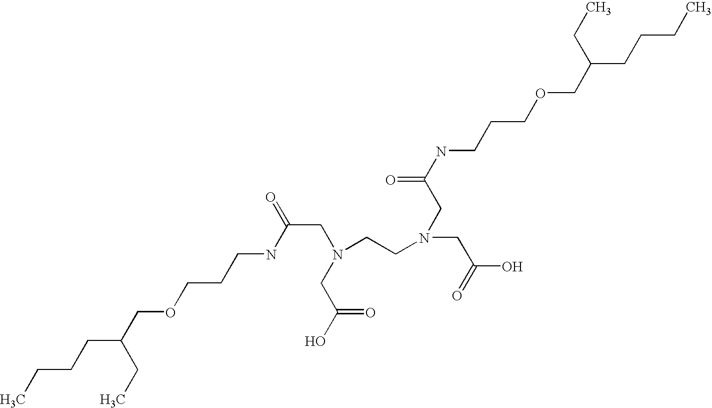

3.03

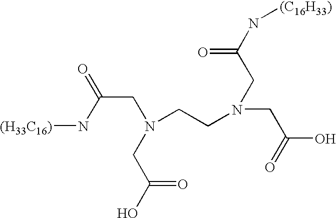

6.10

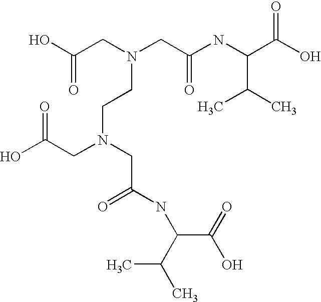

7.20

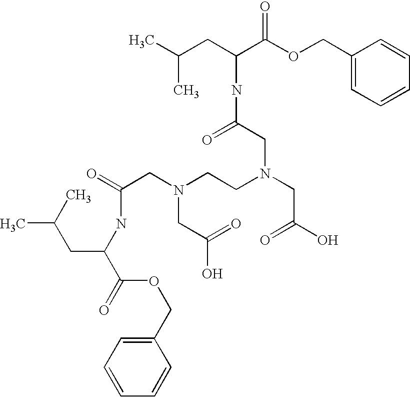

6.00

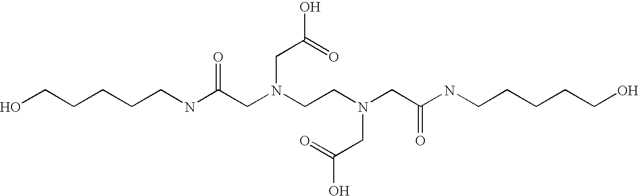

7.80

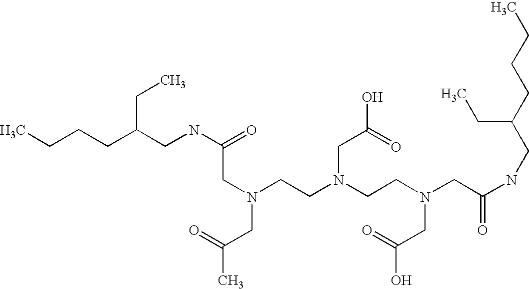

4.46

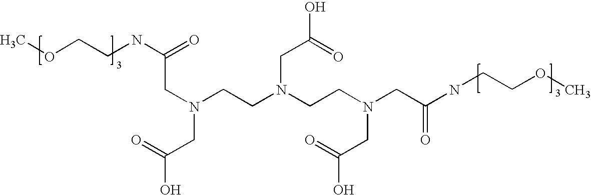

5.84

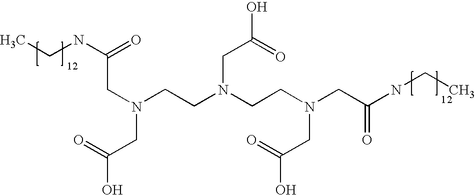

4.10

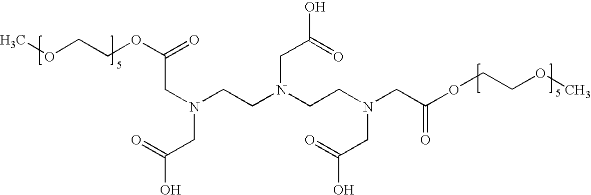

6.66

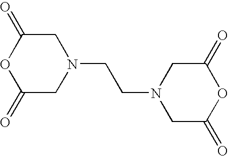

8.00

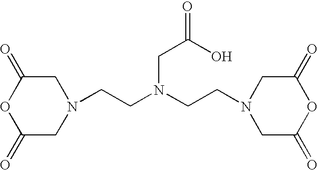

2.14

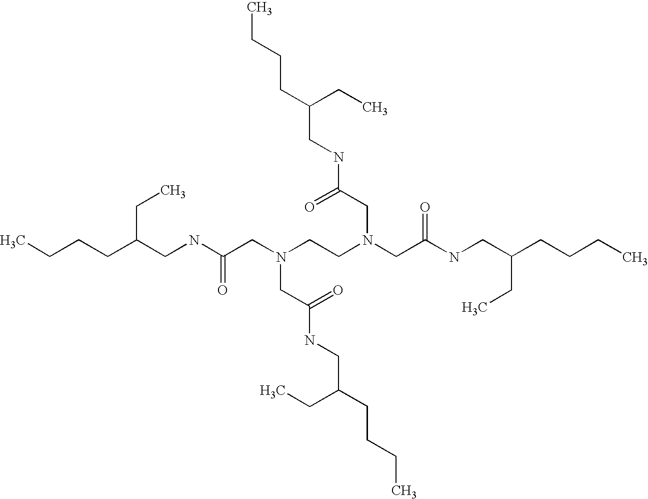

1.99

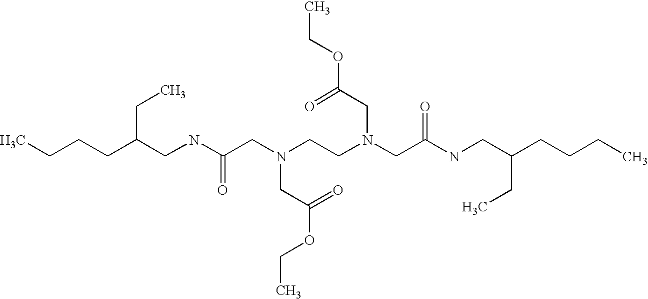

4.10

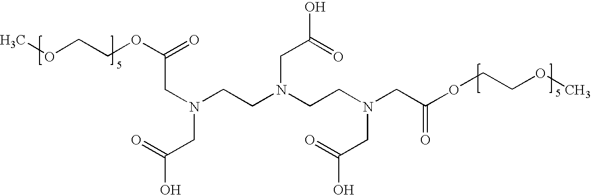

### EXAMPLE 2

90 ml of the monomer with the desired amount of stabilizer were introduced into a 250 ml round-bottomed flask and flushed with the corresponding atmosphere. The flask was heated to the desired storage temperature. Samples were taken at regular intervals. These were investigated with regard to the polymer content by Raman spectroscopy. The curve relative to the unstabilized sample shows the efficiency of the stabilizer system used.

A mixture of N-vinylpyrrolidone (NVP) and pyrrolidone (1:1) was used as a reference, and 25 ppm of PTZ were added thereto. The sample was investigated at 150° C. under an air atmosphere. Rapid polymer formation took place (PVP=polyvinylpyrrolidone). The results are shown in table 4.

| | PVP [% by wt.] |
|---|---|
| Time [min] | 25 ppm PTZ |
| 0 | 0.0 |
| 15 | 4.4 |
| 30 | 9.4 |
| 45 | 13.1 |
| 60 | 16.6 |
| 75 | 19.0 |
| 90 | 21.8 |
| 105 | 23.6 |
| 120 | 25.0 |
| 135 | 27.4 |
| 150 | 29.2 |
| 165 | 30.8 |
| 180 | 32.7 |

A mixture of in each case 25 ppm of free radical scavenger (table 3 from example 1) and 25 ppm of PTZ was added to a mixture of N-vinylpyrrolidone and pyrrolidone (1:1) and heated under identical conditions. No significant differences between the free radical scavengers used in example 1 could be determined. The results of one free radical scavenger (table 3 from example 1, 1st entry) are summarized in table 5. The formation of PVP in the presence of a novel free radical scavenger and PTZ was successfully suppressed.

| | PVP [% by wt.] |
|---|---|
| | 25 ppm PTZ |
| Time [min] | 25 ppm Rs¹⁾ |
| 0 | 0.0 |
| 15 | 0.3 |
| 30 | 0.1 |
| 45 | 0.2 |
| 60 | 0.7 |
| 75 | 0.3 |
| 90 | 0.5 |
| 105 | 0.7 |
| 120 | 1.1 |
| 135 | 2.0 |
| 150 | 1.5 |
| 165 | 2.3 |
| 180 | 2.7 |

¹⁾Rs = Free radical scavenger

On the basis of this example, the effect of different concentrations of PTZ and novel free radical scavenger was then investigated. The results are shown in table 6. From this it is evident that no significant difference in the inhibition of polymerization is detectable from an amount of in each case 25 ppm of PTZ and free radical scavenger.

| | PVP [% by | PVP [% by wt.] | PVP [% by wt.] | PVP [% by wt.] | PVP [% by wt.] |
|---|---|---|---|---|---|
| Time | wt.] | 12.5 ppm PTZ | 25 ppm PTZ | 50 ppm PTZ | 100 ppm PTZ |
| [min] | unstabilized | 12.5 ppm Rs¹⁾ | 25 ppm Rs¹⁾ | 50 ppm Rs¹⁾ | 100 ppm Rs ¹⁾ |
| 0 | 0.6 | 0.4 | 0.0 | 0.1 | 0.3 |
| 15 | 35.2 | 1.1 | 0.3 | 0.0 | 0.0 |
| 30 | 42.1 | 1.9 | 0.1 | 0.3 | 0.2 |
| 45 | 46.2 | 2.9 | 0.2 | 0.1 | 0.2 |
| 60 | 48.1 | 5.2 | 0.7 | 0.4 | 0.6 |
| 75 | 49.7 | 5.6 | 0.3 | 0.5 | 0.7 |
| 90 | 50.1 | 6.5 | 0.5 | 1.0 | 0.6 |
| 105 | 51.2 | 7.7 | 0.7 | 0.8 | 0.9 |
| 120 | 51.8 | 7.9 | 1.1 | 1.2 | 1.0 |
| 135 | 52.6 | 8.7 | 2.0 | 1.4 | 1.5 |
| 150 | 53.2 | 9.0 | 1.5 | 1.1 | 1.9 |
| 165 | 53.3 | 9.3 | 2.3 | 1.3 | 2.1 |
| 180 | 54.1 | 9.7 | 2.7 | 1.8 | 2.7 |

¹⁾Rs = Free radical scavenger

1. A process for stabilizing a polymerizable compound to polymerization during working-up, storages transport, or a combination thereof, comprising adding at least one free radical scavenger which comprises at least two glycine units and at least one amide unit, at least one ester unit, or at least one amide unit and one ester unit, to the polymerizable compound, thereby stabilizing the polymerizable compound to polymerization.

2. The process according to claim 1, wherein the at least one free radical scavenger is a compound of formula (I)
wherein 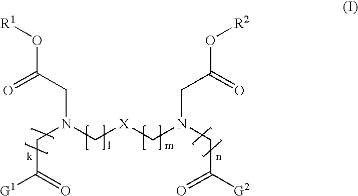
G¹ is NR³R⁴ or OR⁷,
G² is NR⁵R⁶ or OR⁸,
R¹ to R⁶, independently of one another, may be are hydrogen, C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₅-C₁₅-cycloalkylcarbonyl, aryl, arylcarbonyl or heterocycles,
R⁷ and R⁸, independently of one another, are C₁-C₂₀-alkyl, C₁-C₂₀-alkylcarbonyl, C₂-C₂₀-alkenyl, C₂-C₂₀-alkenylcarbonyl, C₂-C₂₀-alkynyl, C₂-C₂₀-alkynylcarbonyl, C₃-C₁₅-cycloalkyl, C₅-C₁₅-cycloalkylcarbonyl, aryl, arylcarbonyl or heterocycles,
X is C₁-C₂₀-alkyl, NCH₂COOR⁹, NR¹⁰, O, S, PR¹¹, Se, SiOR¹²R¹³ or aryl, wherein R⁹ to R¹³, independently of one another, are hydrogen or C₁-C₂₀-alkyl, and
wherein k, l, m and n, independently of one another, may be are numbers from 0 to 20.

3. The process according to claim 2, wherein R¹ and R² are identical and are hydrogen or C₁-C₂₀-alkyl.

4. The process according to claim 2, wherein R³ and R⁵ are identical and are hydrogen, C₁-C₂₀-alkyl or C₁-C₂₀-alkylcarbonyl.

5. The process according to claim 2, wherein R⁴ and R⁶ are identical and are C₁-C₂₀-alkyl, C₁-C₂₀-alkylcarbonyl, aryl, C₂-C₂₀-alkenyl, C₂-C₂₀-alkenylcarbonyl, C₂-C₂₀-alkynyl or C₂-C₂₀-alkynylcarbonyl.

6. The process according to claim 4, wherein R³ and R⁵ are hydrogen and R⁴ and R⁶ are selected from the group consisting of phenyl, benzyl, p-methoxyphenyl, o-hydroxyphenyl, m-hydroxyphenyl p-hydroxyphenyl, 1-hydroxyhexyl, methyl, ethyl, propyl, butyl, ethylene glycol, diethylene glycol, triethylene glycol, ethoxylate having 4 to 10 EO units, ethylenediamine, diethylenetriamine, triethylenetetramine and amino acids.

7. The process according to claim 2, wherein R⁷ and R⁸ are identical and are C₁-C₂₀-alkyl, C₁-C₂₀-alkylcarbonyl, aryl, C₂-C₂₀-alkenyl, C₂-C₂₀-alkenylcarbonyl, C₂-C₂₀-alkynyl or C₂-C₂₀-alkynylcarbonyl.

8. The process according to claim 7, wherein R⁷ and R⁸ are selected from the group consisting of phenyl, benzyl, p-methoxyphenyl, o-hydroxyphenyl, m-hydroxyphenyl p-hydroxyphenyl, 1-hydroxyhexyl, methyl, ethyl, propyl, butyl, ethylene glycol, diethylene glycol, ethoxylate having 4 to 10 EO units, ethylenediamine, diethylenetriamine, triethylenetetramine and amino acids.

9. The process according to claim 2, wherein X is C₁-C₂₀-alkyl or CH₂NCOOR⁹.

10. The process according to claim 1, wherein at least one of the following compounds is employed as the at least one free radical scavenger.

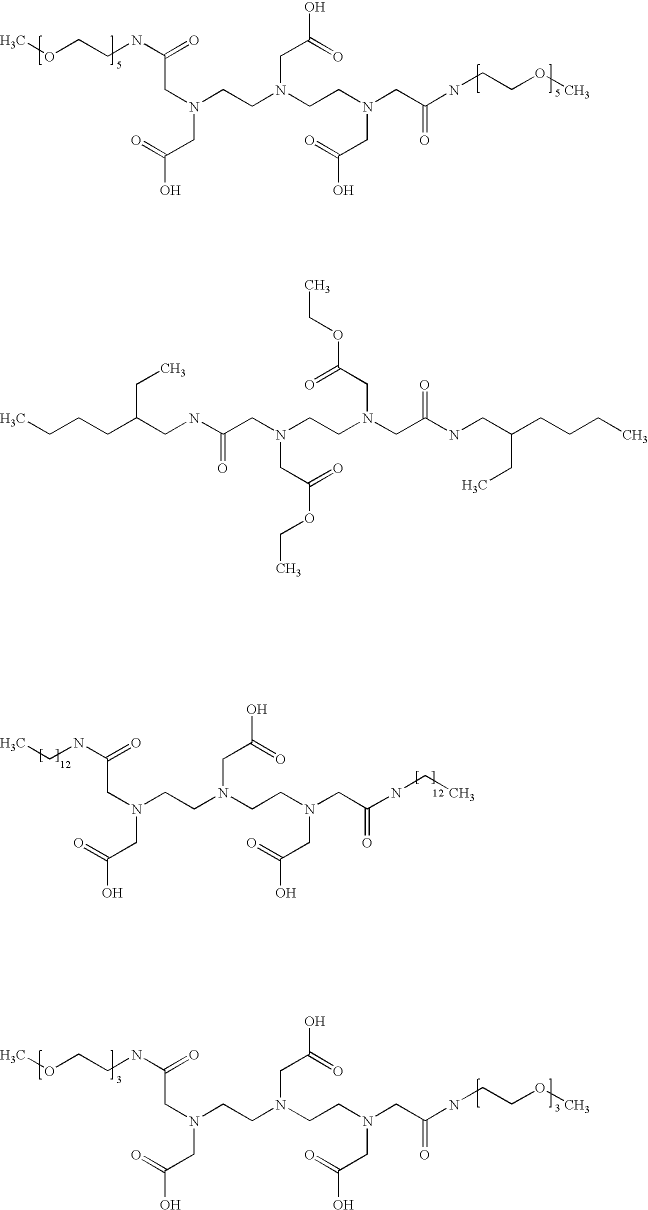

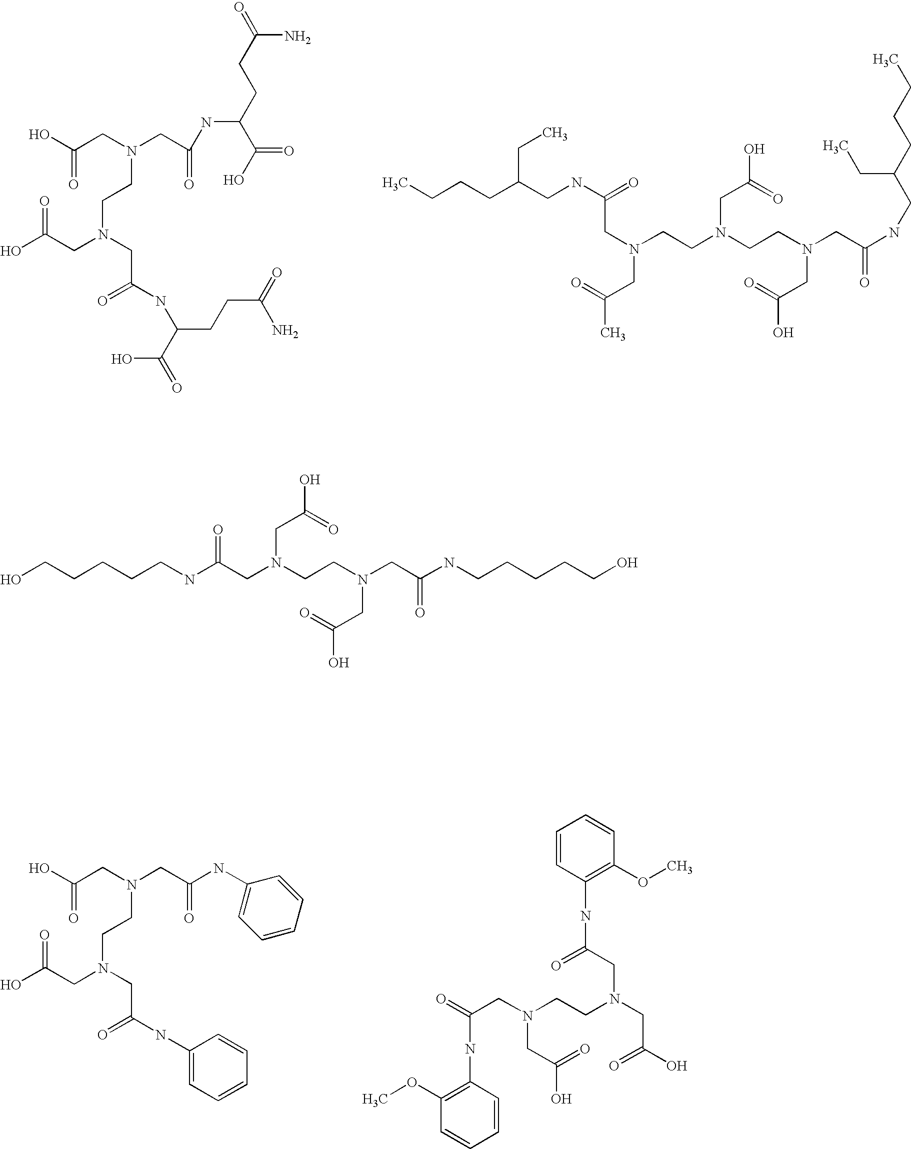

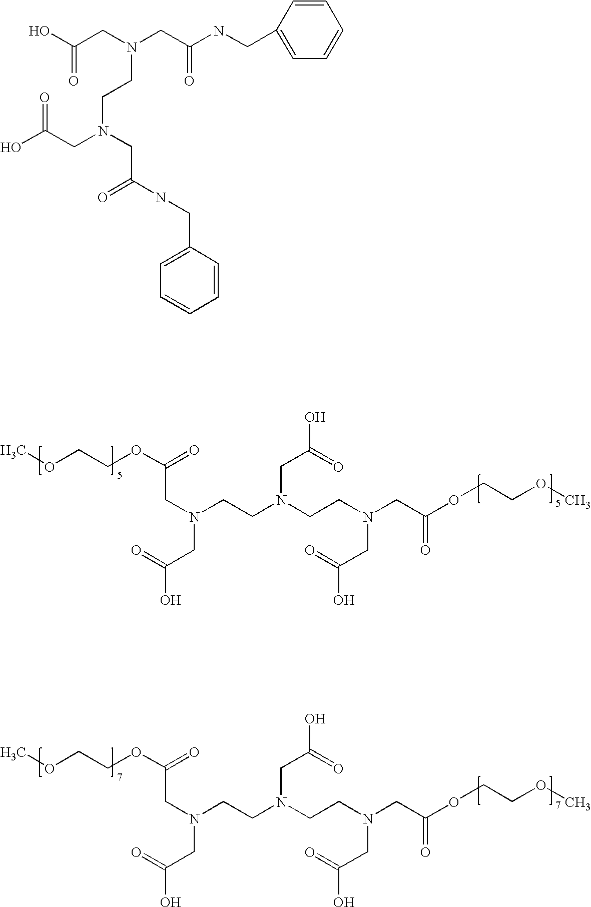

11. The process according to claim 1, wherein from 0.1 to 1,000 ppm, based on the polymerizable compound, of the at least one free radical scavenger is used.

12. The process according to claim 1, further comprising adding at least one costabilizer.

13. The process according to claim 12, wherein the at least one costabilizer is selected from the group consisting of oxygen-containing gases, phenolic compounds, quinines, hydroquinones, N-oxyl compounds, aromatic amines, phenylenediamines, imines, sulfonamides, oximes, hydroxylamines, compounds comprising a urea group, phosphorus-containing compounds, sulfur-containing compounds, complexing agents based on tetraazaannulenes, metal salts and if, mixtures thereof.

14. The process according to claim 12, wherein the at least one costabilizer is selected from the group consisting of phenothiazine, hydroquinone, hydroquinone monomethyl ether, 2,2,6,6-tetramethylpiperidin-N-oxyl, 4-hydroxy-2,2,6,6-tetramethylpiperidin-N-oxyl, 4-oxo-2,2,6,6-tetramethylpiperidin-N-oxyl, N,N′-di-sec-butyl-p-phenylenediamine, cerium(III) acetate, cerium(III) ethylhexanoate, oxygen-containing gases and mixtures thereof.

15. The process according to claim 1, wherein the polymerizable compound comprises at least one ethylenically unsaturated group.

16. The process according to claim 15, wherein the polymerizable compound is selected from the group consisting of the mono-ethylenically unsaturated C₃-C₈-carboxylic acids, di-ethylenically unsaturated C₃-C₈-carboxylic acids, triethylenically unsaturated C₃-C₈-carboxylic acids, C₁-C₂₀-esters of mono-ethylenically unsaturated C₃-C₈-carboxylic acids, C₁-C₂₀-esters of di-ethylenically unsaturated C₁-C₈-carboxylic acids, C₁-C₂₀-esters of tri-ethylenically unsaturated C₃-C₈-carboxylic acids. C₁-C₂₀-amides of mono-ethylenically unsaturated C3-C₈-carboxylic acids, C₁-C₂₀-amides of di-ethylenically unsaturated C₃-C₈-carboxylic acids, C₁-C₂₀-amides of tri-ethylenically unsaturated C₃-C₈-carboxylic acids, C₁-C₂₀-nitriles of mono-ethylenically unsaturated C₃-C₈-carboxylic acids, C₁-C₂₀-nitriles of di-ethylenically unsaturated C₃-C₈-carboxylic acids, C₁-C₂₀-nitriles of tri-ethylenically unsaturated C₃-C₈-carboxylic acids, C₁-C₂₀-anhydrides of mono-ethylenically unsaturated C₃-C₈-carboxylic acids, C₁-C₂₀-anhydrides of di-ethylenically unsaturated C₃-C₈-carboxylic acids. C₁-C₂₀-anhydrides of triethylenically unsaturated C₃-C₈-carboxylic acids, vinyl esters of carboxylic acids comprising up to 20 carbon atoms, vinyl ethers of alcohols comprising from 1 to 10 carbon atoms, vinylaromatics of up to 20 carbon atoms, vinylheteroaromatics of up to 20 carbon atoms, vinyllactams having 3 to 10 carbon atoms in the ring, open-chain N-vinylamide compounds, N-vinylamine compounds, vinyl halides, aliphatic hydrocarbons having 2 to 8 carbon atoms and 1 or 2 double bonds, halogenated hydrocarbons having 2 to 8 carbon atoms and 1 or 2 double bonds, vinylidenes and mixtures of these monomers.

17. The process according to claim 15, wherein the polymerizable compound is selected from the group consisting of mono-ethylenically unsaturated C₃-C₈-carboxylic acids, di-ethylenically unsaturated C₃-C₈-carboxylic acids, triethylenically unsaturated C₃-C₈-carboxylic acids, C₁-C₂₀-esters of mono-ethylenically unsaturated C₁-C₈-carboxylic acids, C₁-C₂₀-esters of di-ethylenically unsaturated C₃-C₈-carboxylic acids, C₁-C₂₀-esters of triethylenically unsaturated C₃-C₈-carboxylic acids, vinyl esters of carboxylic acids comprising up to 20 carbon atoms, vinyl ethers of alcohols comprising 1 to 10 carbon atoms, vinylaromatics of up to 20 carbon atoms, vinylheteroaromatics of up to 20 carbon atoms, vinyllactams having 3 to 10 carbon atoms in the ring, open-chain N-vinylamide compounds, N-vinylamine compounds, and mixtures thereof.

18. The process according to claim 15, wherein the polymerizable compound is selected from the group consisting of (meth)acrylic acid, (meth)acrylates, N-vinylcaprolactam, N-vinylformamide, N-vinylimidazole, N-vinylpyrrolidone, vinylphosphoric acids, N-vinylcarbazole, N,N-divinylethyleneurea, trimethylolpropane triacrylate, ureidomethyl methacrylate, styrene, butadiene, isoprene, and mixtures thereof.

19. A stabilizer mixture comprising
i) at least one free radical scavenger which comprises at least two glycine units and at least one amide unit, at least one ester unit, or at least one amide unit and one ester unit, and
ii) at least one further stabilizer or costabilizer.

20. A mixture comprising the stabilizer mixture according to claim 19 and at least one polymerizable compound.

21. (canceled)
